(19) United States
(12) Patent Application Publication
(10) Pub. No.: US 2026/0041895 A1
(43) Pub. Date: Feb. 12, 2026

(54) BALLOON CATHETERS, BALLOONS THEREFOR, AND METHODS AND DEVICES FOR FORMING CATHETER BALLOONS

(71) Applicant: EDWARDS LIFESCIENCES CORPORATION, Irvine, CA (US)

(72) Inventors: Yidong M. Zhu, Tustin, CA (US); Phu Vinh Le, Garden Grove, CA (US); Roland Patrick Moder, Fountain Valley, CA (US); Tung T. Lam, Irvine, CA (US); Michael R. Bialas, Lake Forest, CA (US)

(21) Appl. No.: 19/361,427

(22) Filed: Oct. 17, 2025

Related U.S. Application Data
(63) Continuation of application No. PCT/US2024/026469, filed on Apr. 26, 2024.
(60) Provisional application No. 63/498,702, filed on Apr. 27, 2023.
(60) Provisional application No. 63/582,308, filed on Sep. 13, 2023.

(51) Int. Cl. A61M 25/10; A61F 2/24

(52) U.S. Cl. CPC A61M 25/1029; A61F 2/2433; A61M 2025/1084; A61M 2025/1086; A61M 2207/10

(57) ABSTRACT

A mold for a catheter balloon can comprise a first mold portion, a second mold portion, and a channel formed on the first mold portion. The first mold portion can comprise a first inner surface and a first outer surface. The second mold portion can comprise a second inner surface and a second outer surface, wherein the first mold portion and the second mold portion form a parting line extending in an axial direction of the mold when the first mold portion and the second mold portion are coupled together. The channel can be formed on the first mold portion parallel to and adjacent the parting line, wherein the channel extends in a radial direction from the first inner surface towards the first outer surface. The mold can be configured to form a balloon comprising features that help facilitate the retrieval and removal of the balloon from a patient's vasculature.

### CROSS REFERENCE TO RELATED APPLICATIONS

This application is a continuation of PCT Patent Application No. PCT/US2024/026469 filed on Apr. 26, 2024, which application claims the benefit of U.S. Provisional Patent Application No. 63/498,702, filed Apr. 27, 2023, and U.S. Provisional Patent Application No. 63/582,308, filed Sep. 13, 2023, all of these applications being incorporated by reference herein in their entireties.

### FIELD

The present disclosure relates to balloon catheters for prosthetic medical devices.

### BACKGROUND

The human heart can suffer from various valvular diseases. These valvular diseases can result in significant malfunctioning of the heart and ultimately require repair of the native valve or replacement of the native valve with an artificial valve. There are a number of known repair devices (for example, stents) and artificial valves, as well as a number of known methods of implanting these devices and valves in humans. Percutaneous and minimally-invasive surgical approaches are used in various procedures to deliver prosthetic medical devices to locations inside the body that are not readily accessible by surgery or where access without surgery is desirable. In one specific example, a prosthetic heart valve can be mounted in a crimped state on the distal end of a delivery apparatus (such as a balloon catheter) and advanced through the patient's vasculature (such as through a femoral artery and the aorta) until the prosthetic heart valve reaches the implantation site in the heart. The prosthetic heart valve is then expanded to its functional size, for example, by inflating a balloon (such as a catheter balloon) on which the prosthetic heart valve is mounted, actuating a mechanical actuator that applies an expansion force to the prosthetic heart valve, or by deploying the prosthetic heart valve from a sheath of the delivery apparatus so that the prosthetic heart valve can self-expand to its functional size.

In some examples, the balloon of the delivery apparatus can tear during an implantation procedure, such as from inadvertent overinflation. A tear in a lateral or circumferential direction across the balloon can result in a portion of the balloon separating from the balloon catheter and/or the balloon becoming caught on the medical implant being implanted with the delivery apparatus, or on another component the delivery apparatus when removing the delivery apparatus from a body of a patient.

Accordingly, a need exists for improved balloons for balloon catheters and methods of fabricating such balloons that can avoid lateral tears if the balloon should fail during a procedure.

### SUMMARY

Described herein are balloon catheters for use in medical procedures. The disclosed balloon catheters can, for example, be configured to tear in an axial direction under pressure from an inflation pressure received by the catheter balloon, thereby simplifying retrieval and removal of torn catheter balloons from the patient's body. As such, the devices disclosed herein can, among other things, overcome one or more of the deficiencies of known balloon catheters. In some examples, balloon catheters disclosed herein can comprise delivery apparatuses that are adapted to delivery and deploy a medical implant (for example, a stent or prosthetic valve) inside a patient's body. In some examples, catheter balloon molds disclosed herein can be configured to form at least a portion (for example, catheter balloons) of any one of the disclosed balloon catheters.

A balloon catheter can comprise a handle and one or more shafts coupled to the handle.

In some examples, a balloon catheter can comprise a catheter balloon mounted on a distal end portion of one of the shafts.

In some examples, the catheter balloon can comprise a main body having a tapered distal end portion, a tapered proximal end portion, and an intermediate portion extending therebetween.

In some examples, the catheter balloon can comprise least one protrusion disposed on an outer surface of the main body. The protrusion can extend in an axial direction along a length of the main body and in a radially outward direction.

In some examples, the main body can comprise a main body sidewall with a first thickness and the protrusion can comprise a protrusion sidewall with a second thickness.

In some examples, the first thickness can be greater than the second thickness.

In some examples, the first thickness can be less than the second thickness.

In some examples, the catheter balloon can be configured to first rupture at the protrusion when the catheter balloon is overinflated.

In some examples, the catheter balloon can further comprise a transition region disposed on the outer surface of the main body and adjacent the protrusion. The catheter balloon can be configured to first rupture at the transition region when the catheter balloon is overinflated.

In some examples, the protrusion can be disposed on the outer surface of the main body at the distal end portion of the main body.

In some examples, the protrusion can be disposed on the outer surface of the main body only at the distal end portion of the main body.

In some examples, the protrusion can be elongated in the axial direction.

In some examples, the catheter balloon can be configured to rupture under a predetermined pressure from an inflation fluid introduced into the catheter balloon.

In some examples, a balloon catheter can comprise a shaft having a proximal end portion and a distal end portion and an inflatable balloon mounted on the distal end portion of the shaft. The balloon can comprise a main body having a tapered distal end portion, a tapered proximal end portion, and an intermediate portion extending therebetween. The balloon can further comprise at least one protrusion disposed only on an outer surface of the distal end portion of the main body of the balloon. The at least one protrusion can extend in an axial direction of the distal end portion of the balloon and in a radially outward direction.

In some examples, a balloon catheter can comprise a shaft and a balloon coupled to a distal end portion of the shaft. The balloon can comprise a main body having a first thickness, and a protrusion extending radially outwards from the main body and in an axial direction along the main body. The protrusion can comprise a sidewall having a second thickness. The first thickness can be greater than the second thickness.

In some examples, a catheter balloon can comprise a first portion extending in a circumferential direction around the catheter balloon and a second portion extending along a length of the first portion and in a radially outward direction. The second portion can be configured to rupture prior to the first portion in an axial direction under a predetermined pressure from an inflation fluid introduced into the catheter balloon.

In some examples, a catheter balloon can comprise a first portion extending in a circumferential direction around the balloon and having a first wall thickness. The catheter balloon can further comprise a second portion extending in an axial direction along a length of the first portion and in a radially outward direction. The second portion can have a second wall thickness, wherein the first wall thickness can be greater than the second wall thickness.

In some examples, a catheter balloon can comprise a main body comprising a first sidewall having a first sidewall thickness. The catheter balloon can further comprise a plurality of protrusions extending radially outwards from the main body and in an axial direction along the main body, wherein each of the plurality of protrusions can comprise a second sidewall having a second sidewall thickness, wherein the first sidewall thickness can be greater than the second sidewall thickness.

In some examples, a catheter balloon can comprise a main body comprising an inner surface, an outer surface, and a first thickness therebetween. The catheter balloon can further comprise at least one axially elongated protrusion extending radially outwards from the outer surface. The axially elongated protrusion can comprise a sidewall having a second thickness, wherein the first thickness can be greater than the second thickness.

In some examples, a catheter balloon can comprise a main body comprising an inner surface, an outer surface, and a first sidewall thickness therebetween. The catheter balloon can further comprise a plurality of weak spots extending radially outwards from the outer surface and disposed along a length of the main body, wherein the each of the plurality of weak spots comprises a sidewall having a second sidewall thickness. The first sidewall thickness can be greater than the second sidewall thickness.

In some examples, a catheter balloon can comprise one or more of the components recited in examples 1-44 and 77 below.

In some examples, a catheter balloon mold can comprise a first mold portion and a second mold portion.

In some examples the catheter balloon mold can comprise a channel formed on the first mold portion.

In some examples, the channel can extend in an axial direction of the mold.

In some examples, the channel can be adjacent a parting line formed by the first mold portion and the second mold portion.

In some examples, the channel can extend in a radial direction from an inner surface of the first mold portion towards an outer surface of the first mold portion.

In some examples, the channel can have a depth in the radial direction from 0.0002″ to 0.005″.

In some examples, the catheter balloon mold can comprise a filler at least partially filling the channel.

In some examples, the filler can completely fill the channel.

In some examples, the filler can only partially fill the channel.

In some examples, the first mold portion can be formed from a first material having a first thermal conductivity, and the filler can be formed from a second material having a second thermal conductivity that is different than the first thermal conductivity.

In some examples, a mold for a catheter balloon can comprise a first mold portion, a second mold portion, and a channel. The first mold portion can comprise a first inner surface and a first outer surface. The second mold portion can comprise a second inner surface and a second outer surface, wherein the first mold portion and the second mold portion can form a parting line extending in an axial direction of the mold when the first mold portion and the second mold portion are coupled together. The channel can be formed on the first mold portion parallel to and adjacent the parting line, wherein the channel can extend in a radial direction from the first inner surface towards the first outer surface.

In some examples, a mold can comprise a body, a trough, and a filler. The body can comprise an inner surface and an outer surface. The body can be formed from a first material having a first thermal conductivity. The trough can be formed on the inner surface of the body. The trough can extend from the inner surface towards the outer surface and in an axial direction of the mold. The filler can at least partially fill the trough, wherein the filler can be formed of a second material having a second thermal conductivity that is different than the first thermal conductivity.

In some examples, a method of forming a catheter balloon can comprise inserting a parison into a mold and blow molding the parison to form the catheter balloon. The mold can comprise a parting line extending in an axial direction of the mold, an inner surface, an outer surface, and at least one channel extending parallel to and adjacent the parting line and in a radial direction from the inner surface towards the outer surface. Blow molding the parison can inflate the parison within the mold and can cause a first portion of the parison to contact the inner surface of the mold and a second portion of the parison to extend into the channel to form a protrusion having a wall thickness that is less than a wall thickness of the first portion.

In some examples, a method of forming a catheter balloon can comprise inserting a parison into a mold comprising an inner surface and an outer surface and at least one gap extending in an axial direction of the mold and in a radial direction from the inner surface towards the outer surface. The method can further comprise blow molding the parison to form the catheter balloon, wherein blow molding the parison can inflate the parison within the mold and can cause a first portion of the parison to contact the inner surface of the mold and a second portion of the parison to extend into the gap to form a protrusion having a wall thickness that is less than a wall thickness of the first portion.

The various innovations of this disclosure can be used in combination or separately. This summary is provided to introduce a selection of concepts in a simplified form that are further described below in the detailed description. This summary is not intended to identify key features or essential features of the claimed subject matter, nor is it intended to be used to limit the scope of the claimed subject matter. The foregoing and other objects, features, and advantages of the disclosure will become more apparent from the following detailed description, claims, and accompanying figures.

### BRIEF DESCRIPTION OF THE DRAWINGS

FIG. 1 is a perspective view of a prosthetic heart valve, according to one example.

FIG. 2 is a perspective view of a delivery apparatus for a prosthetic heart valve, according to one example.

FIG. 3A is a side view of a distal end portion of a balloon catheter comprising a balloon shown in a deflated state, according to a first example.

FIG. 3B is a side view of the distal end portion of the balloon catheter of FIG. 3A showing the balloon in an inflated state.

FIG. 4 is a cross-section of the catheter balloon of FIGS. 3A-3B taken along line A-A of FIG. 3B.

FIG. 5A is a side view of the distal end portion of the balloon catheter of FIGS. 3A-3B showing an axial tear starting to form along a distal end portion of the balloon.

FIG. 5B is a side view of the distal end portion of the balloon catheter similar to FIG. 5A showing the axial tear propagated along a length of the distal end portion of the balloon.

FIG. 5C is a side view of the distal end portion of the balloon catheter similar to FIG. 5A showing the axial tear propagated along a length of an intermediate portion of the balloon.

FIG. 6 is a side view of a distal end portion of a balloon catheter, according to a second example.

FIG. 7 is a side view of a distal end portion of a balloon catheter, according to a third example.

FIG. 8 is a side view of a distal end portion of a balloon catheter, according to a fourth example.

FIG. 9 is a side view of a distal end portion of a balloon catheter, according to a fifth example.

FIG. 10 is a side view of a distal end portion of a balloon catheter, according to a sixth example.

FIG. 11 is a side view of a distal end portion of a balloon catheter, according to a seventh example.

FIG. 12 is a side view of a distal end portion of a balloon catheter, according to an eighth example.

FIG. 13 is a side view of a distal end portion of a balloon catheter, according to a ninth example.

FIG. 14 is a side view of a mold for forming a catheter balloon, according to a first example.

FIG. 15 is a perspective view of a distal mold portion of the mold of FIG. 14.

FIG. 16A is a cross section of the distal mold portion taken along line B-B of FIG. 14 during a first stage of a blow molding process, according to one example.

FIG. 16B is a cross section of the distal mold portion similar to FIG. 16A during a second stage of a blow molding process.

FIG. 17A is a cross section of a distal mold portion during the first stage of a blow molding process, according to another example.

FIG. 17B is a cross section of the distal mold portion similar to FIG. 17A during the second stage of the blow molding process.

FIG. 18 is a cross section of a distal mold portion during the second stage of the blow molding process, according to a third example.

FIG. 19 is a side view of a distal end portion of a balloon catheter, according to a tenth example.

FIG. 20 is a side view of a mold for forming a catheter balloon, according to a second example.

FIG. 21 is a cross-section of the mold taken along line A-A of FIG. 20, according to one example.

FIG. 22 is a cross-section of the mold taken along line B-B of FIG. 20 during a second stage of a blow molding process, according to one example.

FIG. 23 is a cross-section of the mold taken along line C-C of FIG. 20 during the second stage of the blow molding process, according to one example.

FIG. 24 is a side view of the distal end portion of a balloon catheter, according to an eleventh example.

FIG. 25 is a cross-section of the mold for forming a catheter balloon, according to a third example.

FIG. 26 is a cross-section of the mold of FIG. 24 during a second stage of a blow molding process, according to one example.

### DETAILED DESCRIPTION

### General Considerations

For purposes of this description, certain aspects, advantages, and novel features of examples of this disclosure are described herein. The disclosed methods, apparatus, and systems should not be construed as being limiting in any way. Instead, the present disclosure is directed toward all novel and nonobvious features and aspects of the various disclosed examples, alone and in various combinations and sub-combinations with one another. The methods, apparatus, and systems are not limited to any specific aspect or feature or combination thereof, nor do the disclosed examples require that any one or more specific advantages be present or problems be solved.

Although the operations of some of the disclosed examples are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

As used in this application and in the claims, the singular forms “a,” “an,” and “the” include the plural forms unless the context clearly dictates otherwise. Additionally, the term “includes” means “comprises.” Further, the term “coupled” generally means physically, mechanically, chemically, magnetically, and/or electrically coupled or linked and does not exclude the presence of intermediate elements between the coupled or associated items absent specific contrary language.

As used herein, the term “proximal” refers to a position, direction, or portion of a device that is closer to the user and further away from the implantation site. As used herein, the term “distal” refers to a position, direction, or portion of a device that is further away from the user and closer to the implantation site. Thus, for example, proximal motion of a device is motion of the device away from the implantation site and toward the user (for example, out of the patient's body), while distal motion of the device is motion of the device away from the user and toward the implantation site (for example, into the patient's body). The terms “longitudinal” and “axial” refer to an axis extending in the proximal and distal directions, unless otherwise expressly defined.

As used herein, the term “axial direction” refers to a direction that is parallel to a central longitudinal axis of a device, such as a balloon catheter or a catheter balloon.

As used herein, the terms “radial direction” and “lateral direction” refer to a direction that extends radially outward from the central longitudinal axis of the catheter balloon and is perpendicular to the axial direction.

As used herein, the term “circumferential direction” refers to a direction along a circumference of an object (such as a catheter balloon).

As used herein, the term “thickness” refers to a radial thickness of an object (such as a sidewall of a catheter balloon) between an inner circumferential surface and an outer circumferential surface of the object.

As used herein, “e.g.” means “for example,” and “i.e.” means “that is.”

### Overview of the Disclosed Technology

Described herein are examples of balloon catheters that can be used in various medical procedures. In some examples, the disclosed balloon catheters can comprise a delivery apparatus that can be used to navigate a subject's vasculature to deliver an implantable, expandable medical device (for example, a prosthetic heart valve), tools, agents, or other therapy to a location within the body of a subject. Examples of procedures in which the catheters are useful include neurological, urological, gynecological, fertility (for example, in vitro fertilization, artificial insemination), laparoscopic, arthroscopic, transesophageal, transvaginal, transvesical, transrectal, and procedures including access in any body duct or cavity. Particular examples include placing implants, including stents, grafts, embolic coils, and the like; positioning imaging devices and/or components thereof, including ultrasound transducers; and positioning energy sources, for example, for performing lithotripsy, RF sources, ultrasound emitters, electromagnetic sources, laser sources, thermal sources, and the like. In some examples, the disclosed balloon catheters can be used for performing procedures for opening or widening a blood vessel or heart valve annulus, such as an angioplasty or a valvuloplasty.

During a medical procedure involving a balloon catheter, the balloon of the catheter is inflated (such as to deploy a prosthetic valve or another type of implant) by injecting an inflation fluid under pressure into the balloon, and then deflated by withdrawing the inflation fluid from the balloon. Thereafter, the catheter is retracted through an introducer sheath and removed from the patient's body. A catheter balloon sometimes can rupture or tear during the medical procedure, such as if the balloon is inadvertently overinflated, which can complicate retrieval and removal of the catheter from the patient's body. For example, if a balloon forms a tear in the circumferential direction, there may be a greater risk of torn portions of the balloon becoming detached from the balloon catheter. Moreover, tearing of the balloon along its proximal end portion while its distal end portion remains intact can cause the distal end portion to retain inflation fluid and/or blood, making it more difficult to retract the balloon into the introducer sheath.

To address one or more problems of known catheter balloons, it would be desirable to control how a catheter balloon ruptures when subjected to overinflation. For example, it would be desirable to design a catheter balloon such that if it is subjected to overinflation and rupture occurs, the balloon will form one or more axially extending tears, and even more desirably, the one or more axially extending tears will form at least along the distal end portion of the balloon.

### Examples of the Disclosed Technology

Prosthetic valves disclosed herein can be radially compressible and expandable between a radially compressed state and a radially expanded state. Thus, the prosthetic valves can be crimped on or retained by an implant delivery apparatus in the radially compressed state during delivery, and then expanded to the radially expanded state once the prosthetic valve reaches the implantation site. It is understood that the prosthetic valves disclosed herein may be used with a variety of implant delivery apparatuses and can be implanted via various delivery procedures, examples of which will be discussed in more detail later.

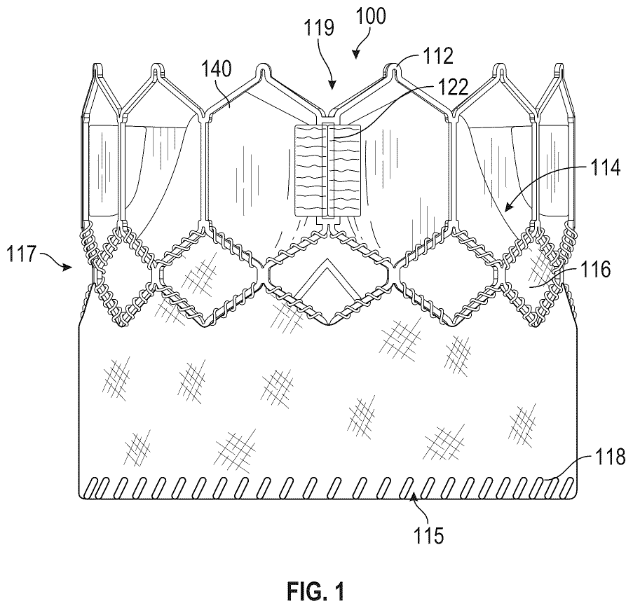

FIG. 1 shows an exemplary prosthetic valve 100, according to one example. Any of the prosthetic valves disclosed herein are adapted to be implanted in the native aortic annulus, although in other examples they can be adapted to be implanted in the other native annuluses of the heart (the pulmonary, mitral, and tricuspid valves). The disclosed prosthetic valves also can be implanted within vessels communicating with the heart, including a pulmonary artery (for replacing the function of a diseased pulmonary valve, or the superior vena cava or the inferior vena cava (for replacing the function of a diseased tricuspid valve) or various other veins, arteries, and vessels of a patient. The disclosed prosthetic valves also can be implanted within a previously implanted prosthetic valve (which can be a prosthetic surgical valve or a prosthetic transcatheter heart valve) in a valve-in-valve procedure.

In some examples, the disclosed prosthetic valves can be implanted within a docking or anchoring device that is implanted within a native heart valve or a vessel. For example, in one example, the disclosed prosthetic valves can be implanted within a docking device implanted within the pulmonary artery for replacing the function of a diseased pulmonary valve, such as disclosed in U.S. Publication No. 2017/0231756, which is incorporated by reference herein. In another example, the disclosed prosthetic valves can be implanted within a docking device implanted within or at the native mitral valve, such as disclosed in PCT Publication No. WO2020/247907, which is incorporated by reference herein. In another example, the disclosed prosthetic valves can be implanted within a docking device implanted within the superior or inferior vena cava for replacing the function of a diseased tricuspid valve, such as disclosed in U.S. Publication No. 2019/0000615, which is incorporated by reference herein.

The prosthetic valve 100 can comprise a frame 112, a valvular structure 114, an inner skirt 116, and a perivalvular outer sealing member or outer skirt 118. The prosthetic valve 100 can comprise an inflow end portion 115 and an outflow end portion 119, and an intermediate portion 117 extending therebetween.

The valvular structure 114 can comprise a plurality of leaflets 140 collectively forming a leaflet structure. In some examples, the valvular structure 114 can comprise three leaflets 140 arranged in a tricuspid arrangement. However, there can be a greater or fewer number of leaflets 140. The leaflets can be secured to one another at their adjacent sides to form commissures 122 of the valvular structure 114. The lower edge of the valvular structure 114 can have an undulating, curved scalloped shape, and can be secured to the inner skirt 116 by sutures (not shown). In some examples, the leaflets 140 can be formed of pericardial tissue (such as bovine pericardial tissue), biocompatible synthetic materials, or other various suitable natural or synthetic materials as known in the art and described in U.S. Pat. No. 6,730,118, which is incorporated by reference herein.

The frame 112 can be made of any of various suitable plastically-expandable materials (for example, stainless steel, etc.) or self-expanding materials (for example, Nitinol) as known in the art. When constructed of a plastically-expandable material, the frame 112 (and thus the valve 100) can be crimped to a radially compressed state on a delivery catheter and then expanded inside a patient by an inflatable catheter balloon or equivalent expansion mechanism. When constructed of a self-expandable material, the frame 112 (and thus the valve 100) can be crimped to a radially compressed state and restrained in the compressed state by insertion into a sheath or equivalent mechanism of a delivery catheter. Once inside the body, the valve can be advanced from the delivery sheath, which allows the valve to expand to its functional size.

Suitable plastically-expandable materials that can be used to form the frames disclosed herein (for example, the frame 112) include, metal alloys, polymers, or combinations thereof. Example metal alloys can comprise one or more of the following: nickel, cobalt, chromium, molybdenum, titanium, or other biocompatible metal. In some examples, the frame 112 can comprise stainless steel. In some examples, the frame 112 can comprise cobalt-chromium. In some examples, the frame 112 can comprise nickel-cobalt-chromium. In some examples, the frame 112 comprises a nickel-cobalt-chromium-molybdenum alloy, such as MP35N™ (tradename of SPS Technologies), which is equivalent to UNS R30035 (covered by ASTM F562-02). MP35N™/UNS R30035 comprises 35% nickel, 35% cobalt, 20% chromium, and 10% molybdenum, by weight.

The inner skirt 116 and/or the outer skirt 118 can be wholly or partly formed of any suitable biological material, synthetic material (for example, any of various polymers), or combinations thereof. In some examples, the skirts 116, 118 can comprise a fabric having interlaced yarns or fibers, such as in the form of a woven, braided, or knitted fabric. In some examples, the fabric can have a plush nap or pile. Exemplary fabrics having a plus nap or pile include velour, velvet, velveteen, corduroy, terrycloth, fleece, etc. In some examples, the skirts 116, 118 can comprise a fabric without interlaced yarns or fibers, such as felt or an electrospun fabric. Exemplary materials that can be used for forming such fabrics (with or without interlaced yarns or fibers) include, without limitation, polyethylene (PET), ultra-high molecular weight polyethylene (UHMWPE), polytetrafluoroethylene (PTFE), expanded polytetrafluoroethylene (ePTFE), polyamide etc. In some examples, the skirts 116, 118 can comprise a non-textile or non-fabric material, such as a film made from any of a variety of polymeric materials, such as PTFE, PET, polypropylene, polyamide, polyetheretherketone (PEEK), polyurethane (such as thermoplastic polyurethane (TPU)), etc. In some examples, the skirts 116, 118 can comprise a sponge material or foam, such as polyurethane foam. In some examples, the skirts 116, 118 can comprise natural tissue, such as pericardium (for example, bovine pericardium, porcine pericardium, equine pericardium, or pericardium from other sources).

### Delivery Apparatus

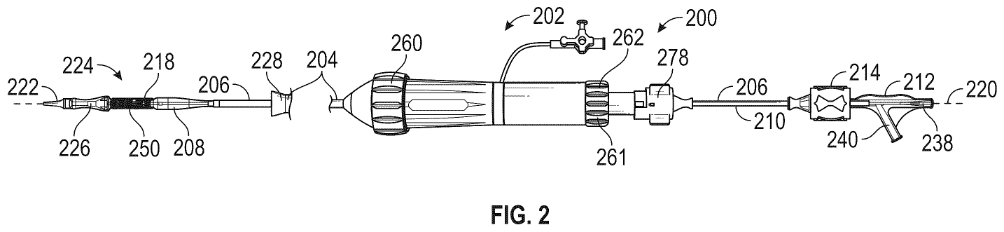

FIG. 2 shows a delivery apparatus 200, according to one example, in the form of a balloon catheter that can be used to implant a prosthetic medical device. In some examples, the delivery apparatus 200 can be used to implant an expandable prosthetic heart valve (for example, the prosthetic heart valve 100 of FIG. 1 and/or any of the other prosthetic heart valves described herein). In some examples, the delivery apparatus 200 is specifically adapted for use in introducing a prosthetic heart valve into a heart.

The delivery apparatus 200 in the illustrated example of FIG. 2 comprises a handle 202 and a steerable, outer shaft 204 extending distally from the handle 202. The delivery apparatus 200 can further comprise an intermediate shaft 206 (which also may be referred to as a balloon shaft) that extends proximally from the handle 202 and distally from the handle 202, the portion extending distally from the handle 202 also extending coaxially through the outer shaft 204. Additionally, the delivery apparatus 200 can further comprise an inner shaft 208 extending distally from the handle 202 coaxially through the intermediate shaft 206 and the outer shaft 204 and proximally from the handle 202 coaxially through the intermediate shaft 206.

The outer shaft 204 and the intermediate shaft 206 can be configured to translate (for example, move) longitudinally, along a central longitudinal axis 220 of the delivery apparatus 200, relative to one another to facilitate delivery and positioning of a prosthetic heart valve at an implantation site in a patient's body.

The intermediate shaft 206 can include a proximal end portion 210 that extends proximally from a proximal end of the handle 202, to an adaptor 212. A rotatable knob 214 can be mounted on the proximal end portion 210 and can be configured to rotate the intermediate shaft 206 around the central longitudinal axis 220 and relative to the outer shaft 204.

The adaptor 212 can include a first port 238 configured to receive a guidewire therethrough and a second port 240 configured to receive fluid (for example, inflation fluid) from a fluid source. The second port 240 can be fluidly coupled to an inner lumen of the intermediate shaft 206.

The intermediate shaft 206 can further include a distal end portion that extends distally beyond a distal end of the outer shaft 204 when a distal end of the outer shaft 204 is positioned away from an inflatable catheter balloon 218 (which also referred to herein as a “balloon”) of the delivery apparatus 200. A distal end portion of the inner shaft 208 can extend distally beyond the distal end portion of the intermediate shaft 206.

The catheter balloon 218 can be coupled to the distal end portion of the intermediate shaft 206.

In some examples, a distal end of the catheter balloon 218 can be coupled to a distal end of the delivery apparatus 200, such as to a nose cone 222 (as shown in FIG. 2), or to an alternate component at the distal end of the delivery apparatus 200 (for example, a distal shoulder). An intermediate portion of the catheter balloon 218 can overlay a valve mounting portion 224 of a distal end portion of the delivery apparatus 200 and a distal end portion of the catheter balloon 218 can overly a distal shoulder 226 of the delivery apparatus 200. The valve mounting portion 224 and the intermediate portion of the catheter balloon 218 can be configured to receive a prosthetic heart valve in a radially compressed state. For example, as shown schematically in FIG. 2, a prosthetic heart valve (which can be one of the prosthetic heart valves described herein) can be mounted around the catheter balloon 218, at the valve mounting portion 224 of the delivery apparatus 200.

The balloon shoulder assembly, including the distal shoulder 226, is configured to maintain the prosthetic heart valve 250 (or other prosthetic medical device) at a fixed position on the catheter balloon 218 during delivery through the patient's vasculature.

The outer shaft 204 can include a distal tip portion 228 mounted on its distal end. The outer shaft 204 and the intermediate shaft 206 can be translated axially relative to one another to position the distal tip portion 228 adjacent to a proximal end of the valve mounting portion 224, when the prosthetic heart valve 250 is mounted in the radially compressed state on the valve mounting portion 224 (as shown in FIG. 2) and during delivery of the prosthetic heart valve to the target implantation site. As such, the distal tip portion 228 can be configured to resist movement of the prosthetic heart valve 250 relative to the catheter balloon 218 proximally, in the axial direction, relative to the catheter balloon 218, when the distal tip portion 228 is arranged adjacent to a proximal side of the valve mounting portion 224.

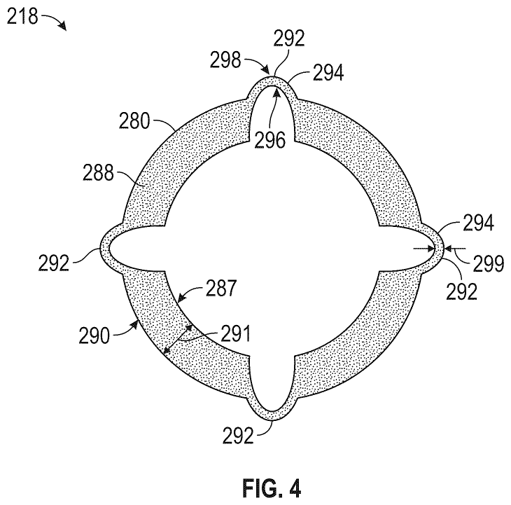

An annular space can be defined between an outer surface of the inner shaft 208 and an inner surface of the intermediate shaft 206 and can be configured to receive fluid from a fluid source via the second port 240 of the adaptor 212. The annular space can be fluidly coupled to a fluid passageway formed between the outer surface of the distal end portion of the inner shaft 208 and an inner surface 287 (FIG. 4) of the catheter balloon 218. As such, fluid from the fluid source can flow to the fluid passageway from the annular space to inflate the catheter balloon 218 and radially expand and deploy the prosthetic heart valve 250.

An inner lumen of the inner shaft can be configured to receive a guidewire therethrough, for navigating the distal end portion of the delivery apparatus 200 to the target implantation site.

The handle 202 can include a steering mechanism configured to adjust the curvature of the distal end portion of the delivery apparatus 200. In the illustrated example, for example, the handle 202 includes an adjustment member, such as the illustrated rotatable knob 260, which in turn is operatively coupled to the proximal end portion of a pull wire. The pull wire can extend distally from the handle 202 through the outer shaft 204 and has a distal end portion affixed to the outer shaft 204 at or near the distal end of the outer shaft 204. Rotating the knob 260 can increase or decrease the tension in the pull wire, thereby adjusting the curvature of the distal end portion of the delivery apparatus 200. Further details on steering or flex mechanisms for the delivery apparatus can be found in U.S. Pat. No. 9,339,384, which is incorporated by reference herein.

The handle 202 can further include an adjustment mechanism 261 including an adjustment member, such as the illustrated rotatable knob 262, and an associated locking mechanism including another adjustment member, configured as a rotatable knob 278. The adjustment mechanism 261 is configured to adjust the axial position of the intermediate shaft 206 relative to the outer shaft 204 (for example, for fine positioning at the implantation site). Further details on the delivery apparatus 200 can be found in PCT Publication No. WO2022/046585, which is incorporated by reference herein.

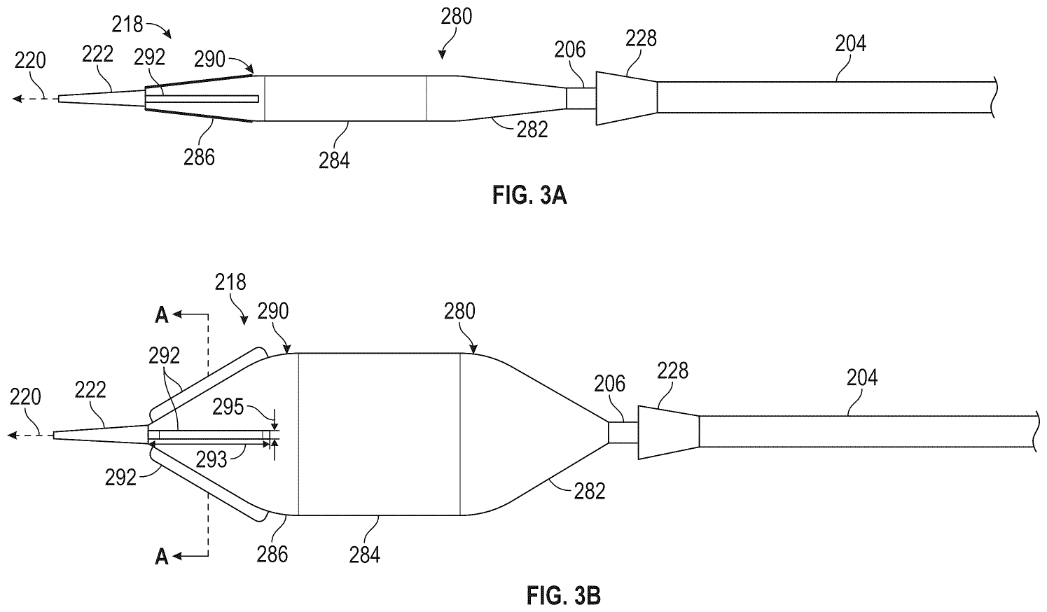

FIGS. 3A-3B show a side view of the catheter balloon 218, according to one example. The catheter balloon 218 can comprise an inflatable main body 280 configured to be inflatable between a deflated state (FIG. 3A) and an inflated state (FIG. 3B). The main body 280 can comprise a proximal end portion 282, a distal end portion 286 distally disposed relative to the proximal end portion 282, and an intermediate portion 284 extending therebetween. In some examples, at least one of the proximal end portion 282 and the distal end portion 286 of the catheter balloon 218 can be tapered. In some examples, at least one of the proximal end portion 282 and the distal end portion 286 of the catheter balloon 218 can comprise a frustoconical shape that tapers away from the intermediate portion 284. The proximal end portion 282 can be coupled to a shaft (such as the intermediate shaft 206) of the delivery apparatus 200. The intermediate portion 284 can be configured to receive a prosthetic heart valve (such as the prosthetic heart valve 100) in a radially compressed state. The distal end portion 286 can be connected to the nose cone 222 or another distal end component of the delivery apparatus 200 (such as a distal shoulder). In some examples, the proximal end portion 282, the intermediate portion 284, and the distal end portion 286 can be integrally formed as a single component.

The main body 280 can further comprise a sidewall 288 (which is also referred to herein a “wall,” a “main body wall,” or a “main body sidewall”) extending in the circumferential direction around the catheter balloon 218 and extending between the proximal end portion 282 and the distal end portion 286. The sidewall 288 can comprise a circumferential inner surface 287 (FIG. 4) that defines an interior of the catheter balloon 218 and circumferential outer surface 290 disposed radially outward of the inner surface 287. The inner surface 287 can define an inner cavity of the main body 280 that is configured to receive an inflation fluid for inflating the main body 280 when the catheter balloon 218 is mounted to the delivery apparatus 200. The sidewall 288 further comprises a thickness in the radial direction (which is also referred to as a “wall thickness,” a “sidewall thickness,” of a “main body thickness”) between the inner surface 287 and the outer surface 290. In some examples, the sidewall 288 can have a uniform circumferential thickness and/or a uniform longitudinal thickness.

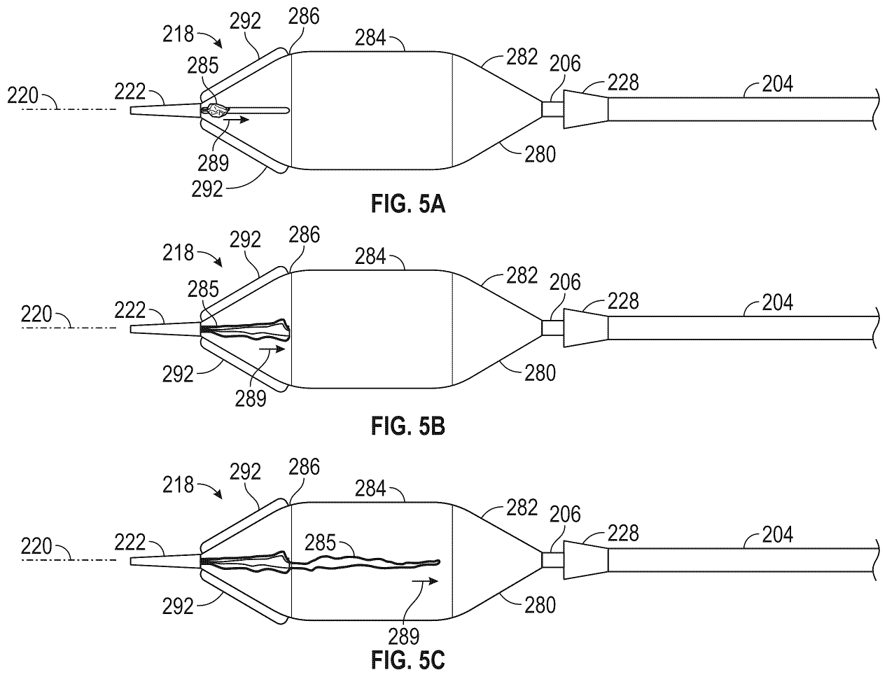

The catheter balloon 218 can further comprise at least one protrusion 292 (which is also referred to herein as a “weak spot” or a “weakened region”) configured to rupture or tear prior to the rest of the catheter balloon 218 (such as the main body 280). The protrusion 292 can be configured to form an axial tear 285 (FIGS. 5A-5C) in the catheter balloon 218 that propagates in the longitudinal or axial direction when the catheter balloon 218 is overinflated. In some examples, the protrusion 292 can be integrally formed with the main body 280. In some examples, the axial tear 285 can be configured to form when the catheter balloon 218 is overinflated to a predetermined pressure from an inflation fluid introduced into the catheter balloon 218.

The protrusion 292 can extend radially outward from the outer surface 290 of the main body 280. The protrusion 292 can extend axially along a length of the main body 280. In some examples, the protrusion 292 can extend along the entire length of the main body 280 from the proximal end portion 282 to the distal end portion 286. In some examples, the protrusion 292 can be disposed on or extend from an outer surface (such as the outer surface 290) of the distal end portion 286 to encourage tears along the distal end portion 286 of the catheter balloon 218. In some examples, the protrusion 292 can be disposed only on the outer surface along the distal end portion 286 of the main body 280. In some examples, the protrusion 292 can extend along an entire axial length of the distal end portion 286. In some examples, the protrusion 292 can be disposed on the outer surface of any of the proximal end portion 282, the intermediate portion 284, and the distal end portion 286.

The protrusion 292 can further comprise a length 293 in the axial direction and a width 295 in the circumferential direction. In some examples, the protrusion 292 can be axially elongated, in that the length 293 of the protrusion 292 is greater than the width 295 of the protrusion 292. Since the protrusion 292 is configured to rupture or tear before the rest of the catheter balloon 218, any resulting tear may be more likely to propagate in the axial direction along the length of the protrusion 292 if the protrusion 292 is axially elongated. In some examples, the length 293 of the protrusion 292 can equal the axial length of the distal end portion 286, such that the protrusion extends along the entire length of the distal end portion 286.

As shown in FIG. 3A, the protrusion 292 can be configured to collapse radially inwards when the catheter balloon 218 is in the deflated state, thereby minimizing the outer diameter of the catheter balloon 218 when the catheter balloon 218 is advanced through the patient's vasculature. In some examples, the protrusion 292 can be flush with the outer surface 290 of the main body 280 when the catheter balloon 218 is in the deflated state. As shown in FIG. 3B, the protrusion can be configured to radially expand when the catheter balloon 218 is in the inflated state.

FIG. 4 shows an annular cross-section of the catheter balloon 218 taken along line A-A of FIG. 3B. In the illustrated example, the catheter balloon 218 comprises four protrusions 292. However, the catheter balloon 218 can comprise any suitable number of protrusions 292, including one, two, three, four, five, six, seven, eight, or more protrusions 292. In some examples where the catheter balloon comprises a plurality of protrusions 292, the protrusions 292 can be evenly spaced apart on the main body 280 in the circumferential direction. In some examples, the protrusions 292 can be unevenly spaced apart on the main body 280 in the circumferential direction to encourage ruptures or tears to first form in a particular radial direction.

The protrusion 292 can include a sidewall 294 (which is also referred to herein as a “protrusion wall” or a “protrusion sidewall”) comprising an inner surface 296, an outer surface 298 disposed radially outward of the inner surface 296, and a thickness 299 (which is also referred to as a “protrusion thickness”) in the radial direction therebetween. In some examples, the thickness 299 of the protrusion 292 can be less than the thickness 291 (which is also referred to herein as a “sidewall thickness”) between the inner surface 287 and the outer surface 290 of the main body 280. In such examples, since the relatively thinner protrusion 292 can be weaker or less resistant to tearing than the relatively thicker main body 280, the catheter balloon 218 may be more likely to rupture first at or along the protrusion 292. In FIG. 4 and the figures described herein, the differences between the sidewall thickness 291 and the protrusion thickness 299 as shown may be exaggerated (larger than actual) for the purposes of illustration and clearly showing the relative sidewall thicknesses. In some examples, the protrusion 292 can have a semi-elliptical or a semicircular axial cross section. However, in some examples, the protrusion 292 can have an axial cross-section of any suitable shape.

FIGS. 5A-5C show a side view of the catheter balloon 218 experiencing an axial tear 285. As shown in FIG. 5A, the axial tear 285 can first form in the protrusion sidewall 294 when the catheter balloon 218 is overinflated. The initial tear 285 may be more likely to form in the protrusion sidewall 294, rather than in the main body sidewall 288, when the catheter balloon 218 is overinflated to a predetermined pressure from an inflation fluid introduced into the catheter balloon 218 because the protrusion sidewall 294 may be thinner and consequently weaker than the main body sidewall 288. As shown in FIG. 5B, the axial tear 285 can propagate in the axial direction (for example, in the direction of arrow 289) along the axial length of the protrusion 292. In some examples, at least one of the ends of the axial tear 285 can be coterminous with at least one of the axial ends of the protrusion 292. In some examples, such as the example shown in FIG. 5C, the axial tear 285 can continue to propagate in the axial direction past the axial ends of the protrusion 292. Even if the axial tear 285 continues in the proximal direction along the intermediate portion 284, the proximal end portion 282 of the main body 280 can remain intact and connected to the shaft 206 of the delivery apparatus 200. Although not shown, one or more of the other protrusions 292 also can form axial tears 285 if the catheter balloon 218 is overinflated. The one or more protrusions 292 are advantageous in that they can prevent or minimize the formation of circumferential tears, which can pose a risk of a portion of the catheter balloon 218 separating from the delivery apparatus 200. Moreover, by virtue of forming one or more axial tears 285 at least along the distal end portion 286 of the balloon 218, any inflation fluid remaining in the catheter balloon 218 after rupture can be forced or squeezed out of the catheter balloon 218 as the catheter balloon 218 is retracted proximally back into an introducer sheath while removing the delivery apparatus 200 from the patient's body.

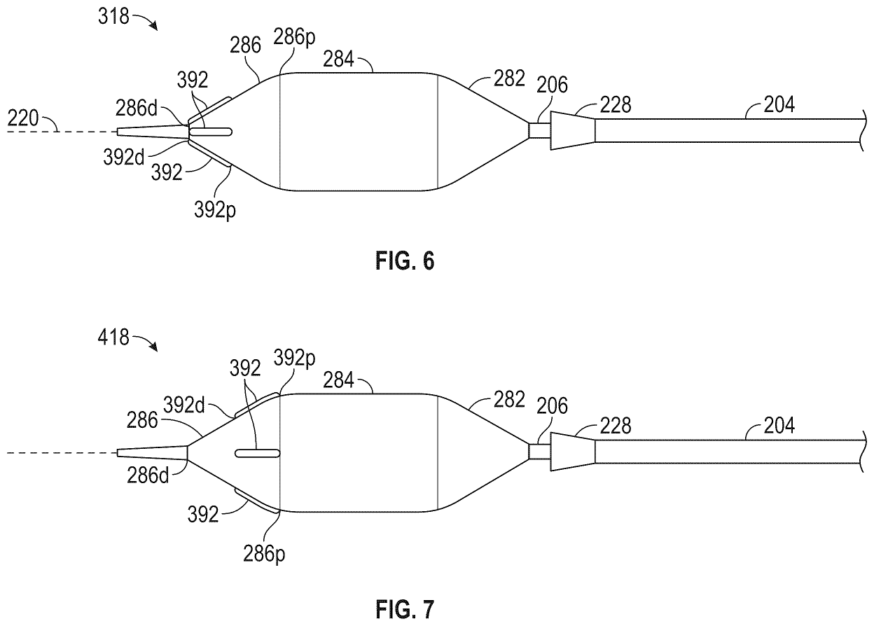

FIG. 6 shows a side view of a catheter balloon 318 with at least one protrusion 392. The protrusion 392 can comprise a proximal end 392p and a distal end 392d. One exemplary difference between the catheter balloon 318 and the catheter balloon 218 of FIGS. 3A-3B is that the protrusion 392 can be axially shorter than the protrusion 292 of FIGS. 3A-3B and can extend along about half the entire axial length of the distal end portion 286 of the main body 280. For example, the distal end 392d of the protrusion 392 can be axially aligned with a distal-most end 286d of the distal end portion 286 of the main body 280. The protrusion 392 can extend partially along the axial length of the distal end portion 286, such that the proximal end 392p of the protrusion 392 can be distally disposed relative to a proximal-most end 286p of the distal end portion 286.

FIG. 7 shows a side view of a catheter balloon 418. One exemplary difference between the catheter balloon 418 and the catheter balloon 318 of FIG. 6 is that the proximal end 392p of the protrusion 392 can be axially aligned with the proximal-most end 286p of the distal end portion 286, and the distal end 392d of the protrusion 392 can be proximally disposed relative to the distal-most end 286d of the distal end portion 286.

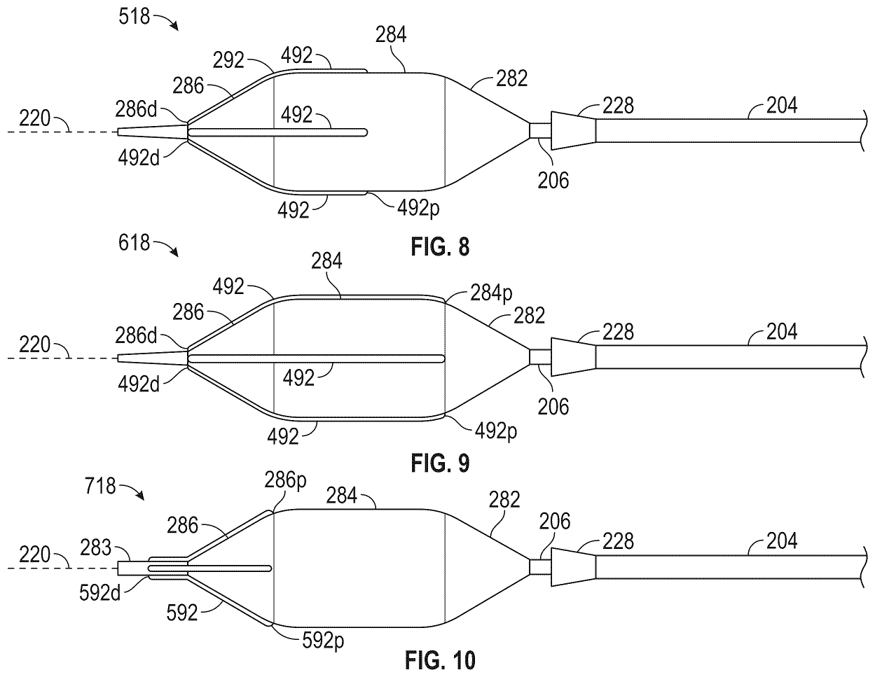

FIG. 8 shows a side view of a catheter balloon 518, wherein the catheter balloon 518 can comprise at least one protrusion 492. One exemplary difference between the catheter balloon 518 and the catheter balloon 218 of FIGS. 3A-3B is that the protrusion 492 can be longer than the protrusion 292 of FIGS. 3A-3B and extend along at least a portion of the length of the distal end portion 286 and a portion of the length of the intermediate portion 284. For example, a distal end 492d of the protrusion 492 can be axially aligned with the distal-most end 286d of the distal end portion 286. The protrusion 492 can extend along the entire axial length of the distal end portion 286 and along a portion of the length of the intermediate portion 284, such that a proximal end 492p of the protrusion 492 can be disposed on an outer surface of the intermediate portion 284. In some examples, disposing at least a portion of the protrusion 492 on the intermediate portion 284 can help control the direction of an axial tear (such as axial tear 285) as the tear propagates in the intermediate portion 284.

FIG. 9 shows a side view of a catheter balloon 618. One exemplary difference between the catheter balloon 618 and the catheter balloon 518 of FIG. 8 is that the protrusion 492 can extend along the entire length of the distal end portion 286 and along the entire length of the intermediate portion 284. For example, the distal end 492d of the protrusion 492 can align with the distal end 286d of the distal end portion 286. The protrusion 492 can extend along the entire axial length of the distal end portion 286 and along the entire axial length of the intermediate portion 284, such that the proximal end 492p of the protrusion 492 can be axially aligned with a proximal end 284p of the intermediate portion 284.

FIG. 10 shows a side view of a catheter balloon 718. One exemplary difference between the catheter balloon 718 and the catheter balloons 518, 618 of FIGS. 8-9 is that the catheter balloon 718 can further comprise a distal stalk 283 that extends in the distal direction from the distal end portion 286. The distal stalk 283 can comprise a cylindrical portion of the catheter balloon 718. The distal stalk 283 can be configured to be coupled to the nose cone 222 (which is omitted in FIG. 10 to show the distal stalk 283). In some examples, the nose cone 222 can be configured to fit over the distal stalk 283. At least one protrusion 592 can extend along the length of the distal end portion 286 and along a length of the distal stalk 283. In some examples, a proximal end 592p of the protrusions 592 can be axially aligned with the proximal-most end 286p of the distal end portion 286 and a distal end 592d of the protrusion 592 can be disposed on an outer surface of the distal stalk 283. In some examples, the protrusion 592 can extend along only a portion of the entire length of the distal stalk 283. In some examples, the protrusion 592 can extend along the entire length of the distal stalk 283.

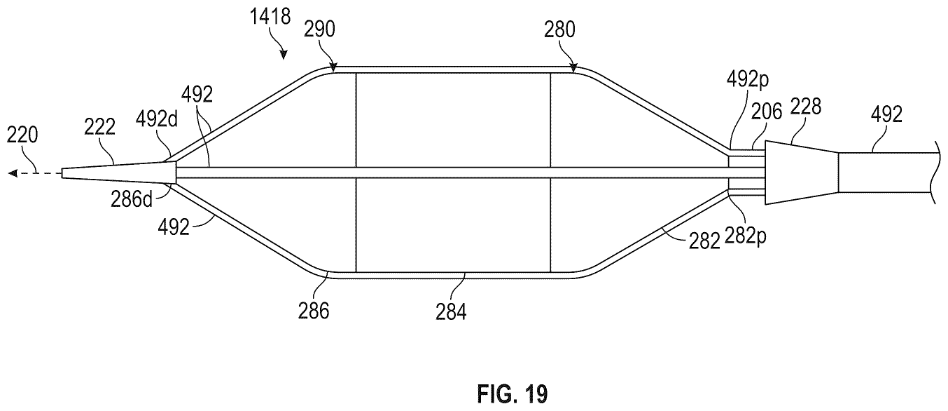

FIG. 19 shows a side view of a catheter balloon 1418. One exemplary difference between the catheter balloon 1418 and the catheter balloon 618 of FIG. 8 is that the protrusions 492 can extend along the entire length of the distal end portion 286, the entire length of the intermediate portion 284, and the entire length of the proximal end portion 282. For example, the distal end 492d of the protrusion 492 can align with the distal end 286d of the distal end portion 286. The protrusion 492 can extend along the entire axial length of the distal end portion 286, along the entire axial length of the intermediate portion 284, along the entire axial length of the proximal end portion 282, and along a proximal end portion of the intermediate shaft 206. In some examples, a proximal end of the protrusion 492 can be axially aligned with the proximal end 282p of the proximal end portion 282.

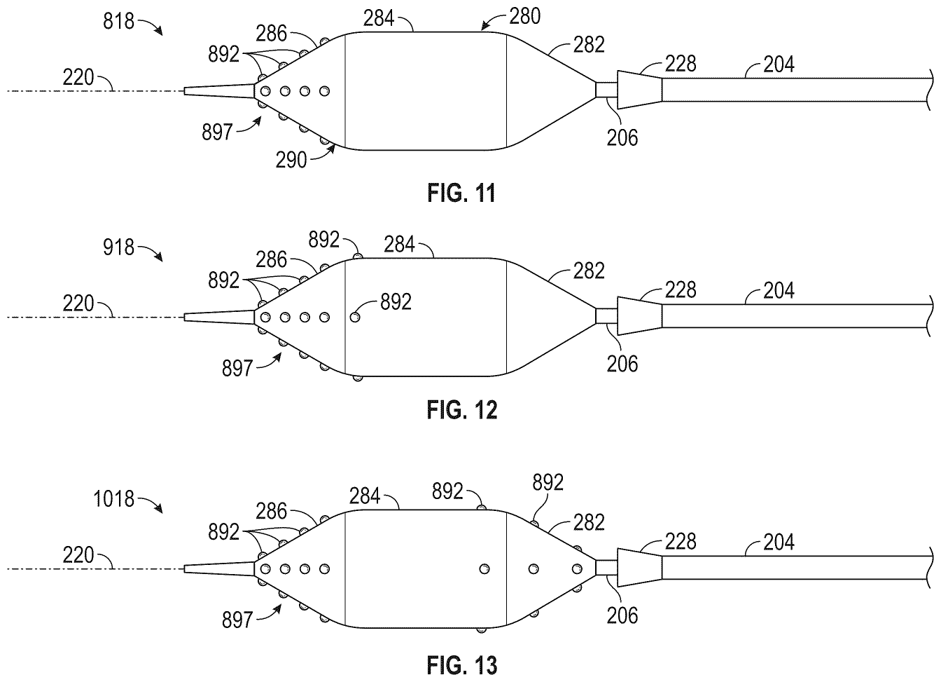

FIG. 11 shows a side view of a catheter balloon 818. The catheter balloon 818 can comprise the main body 280 and at least one protrusion 892 configured to rupture or tear prior to the rest of the catheter balloon 818 (such as the main body 280). Although the protrusion 892 is illustrated as having a hemispherical shape, other examples of the protrusion 892 can have a hemispheroidal shape with an elliptical cross-section. In some examples, the protrusion 892 can be integrally formed with the main body 280.

The protrusion 892 can extend radially outward from the outer surface 290 of the main body 280. The protrusion 892 can comprise a sidewall with an inner surface, an outer surface disposed radially outward of the inner surface, and a thickness therebetween. In some examples, the thickness of the protrusion 892 can be less than the thickness 291 of the main body 280. In some examples, the thickness of the protrusion 892 can be greater than the thickness 291 of the main body 280.

In some examples where the catheter balloon 818 comprises a plurality of protrusions 892, the protrusions 892 can be arranged in one or more groups of plural protrusions 892 arrayed in the axial direction to promote axial tearing of the catheter balloon 818. Since the catheter balloon 818 is configured to first rupture at the protrusions 892, any ruptures or tears can propagate between the axially arranged protrusions 892 in the axial direction. In some examples, the catheter balloon 818 can comprise at least one array 897 of a plurality of protrusions 892 arranged along the axial length of the main body 280. In some examples, the catheter balloon 818 can comprise a plurality of arrays 897 arranged circumferentially around the main body 280. As illustrated in FIG. 11, four arrays 897 of protrusions 892 can be disposed on the outer surface 290 of the distal end portion 286 and spaced circumferentially around the distal end portion 286. In some examples, the arrays 897 can be disposed only on the distal end portion 286 of the main body 280. Each array 897 can comprise, for example, four protrusions 892 aligned along the axial length of the distal end portion 286. In some examples, each array can have more or less than four protrusions 892. In some examples, the protrusions 892 in each array can be evenly spaced in the axial direction or unevenly spaced in the axial direction. Since axial tears are more likely to propagate in thinner or weaker areas of the catheter balloon 818, arranging the protrusions 892 in the axial direction may promote the axial propagation of ruptures or tears (such as axial tear 285). In some examples, the protrusions 892 can be disposed only on the outer surface of the distal end portion 286 because concentrating the protrusions 892 at the distal end portion 286 encourages distal ruptures. However, any suitable number of protrusions 892 may be arranged in any suitable pattern to promote desirable modes of catheter balloon rupturing.

FIG. 12 shows a side view of a catheter balloon 918. One exemplary difference between the catheter balloon 918 and the catheter balloon 818 of FIG. 11 is that at least one of the protrusions 892 is disposed on the intermediate portion 284. In some examples, the protrusion 892 on the intermediate portion 284 can be axially aligned with at least one other protrusion 892 on the distal end portion 286.

FIG. 13 shows a side view of a catheter balloon 1018. One exemplary difference between the catheter balloon 1018 and the catheter balloons 818, 918 of FIGS. 11 and 12 is that at least one of the protrusions 892 is disposed on the proximal end portion 282. In some examples, the protrusion 892 on the proximal end portion 282 can be axially aligned with at least one other protrusion 892 on the distal end portion 286.

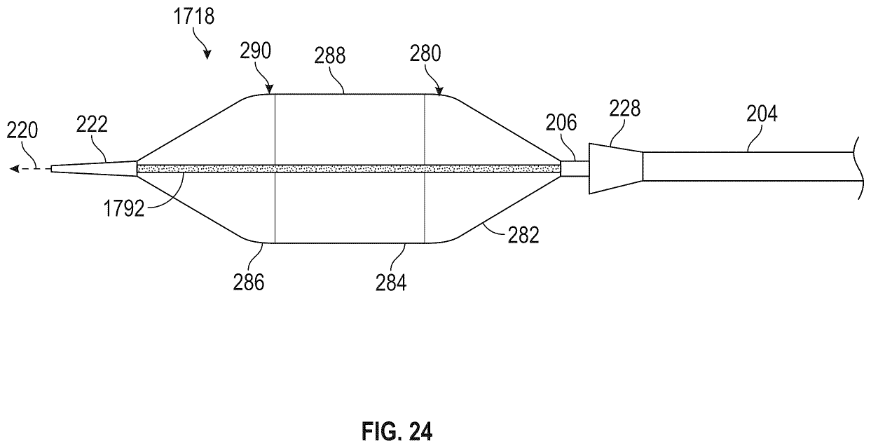

FIG. 24 shows a side view of a catheter balloon 1718. The catheter balloon 1718 can comprise a main body 280. The main body 280 can comprise a sidewall 288, wherein the sidewall 288 can be formed from a material having a first yield strength. The main body 280 can further comprise at least one axial strip 1792, which can replace a portion of the sidewall 288. The axial strip 1792 can extend in the direction of the central longitudinal axis 220 (in other words, the axial direction), can extend from the inner surface to the outer surface of the main body 280, and can extend along only a portion of the circumference of the catheter balloon 1718. The axial strip 1792 can extend along the entire length of the balloon 1718, as shown, or partially along the length of the catheter balloon 1718. The axial strip 1792 can be formed from a material having a second yield strength that is different than the first yield strength. In some examples, the second yield strength can be less than the first yield strength. For example, the sidewall 288 can be formed from a first material having a first yield strength and the axial strip 1792 can be formed from a second, different material having a second, lesser yield strength. In another example, the main body 280 can be formed from a polymer having a first cross-linking density, while the axial strip 1792 can be formed from the same polymer having a second, lesser cross-linking density and a second, lesser yield strength. The stippling in the axial strip 1792 represents that the axial strip 1792 has a different yield strength than the sidewall 288. In such examples, the axial strip 1792 can be configured to tear before the sidewall 288 of the main body 280 to beneficially promote the formation of an axial tear in the catheter balloon 1718.

Although the catheter balloon 1718 is illustrated as comprising one axial strip 1792, the catheter balloon 1718 can comprise any number (for example, two, three, four, five, six, etc.) of axial strips 1792.

In some examples, the axial strip 1792 can be flush with the inner surface 287 of the main body 280 and/or flush with the outer surface 290 of the main body 280. Thus, in some examples, the axial strip 1792 can have the same thickness in the radial direction as the sidewall 288. However, in some examples, the axial strip 1792 can protrude outwards from the sidewall 288. In some examples, the axial strip 1792 can have a lesser thickness in the radial direction than the sidewall 288 of the main body 280.

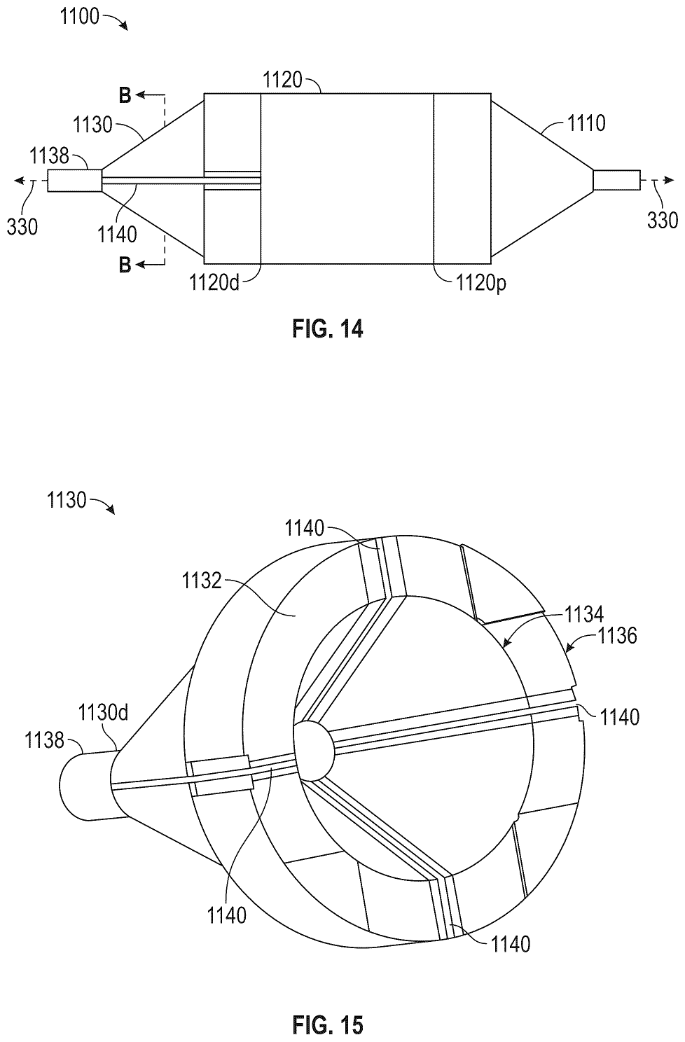

FIG. 14 shows a side view of a mold 1100 (which is also referred to herein as a “blow mold”) for making a catheter balloon, according to one example. The mold 1100 can be configured to form any of the catheter balloons disclosed in this application during a blow molding process. The mold 1100 can comprise a proximal mold portion 1110 (which is also referred to herein as a “proximal mold body”), an intermediate mold portion 1120 (which is also referred to as an “intermediate mold body”), and a distal mold portion 1130 (which is also referred to herein as a “distal mold body”) disposed along a central longitudinal axis 330. The intermediate mold portion 1120 can comprise a proximal end 1120p configured to be coupled to or abut a distal end of the proximal mold portion 1110 and a distal end 1120d configured to be coupled to or abut a proximal end of the distal mold portion 1130. During a blow molding process, a parison 1142 (FIG. 16A) is placed inside the mold 1100 and inflated. It should be understood that “inflating” the parison 1142 (or the inflation of any other parison disclosed herein, such as parisons 1642 and 1942) can comprise steps such as heating, pressurizing, and stretching the parison 1142 within the mold 1100 to make the catheter balloon. Furthermore, it should be understood that the mold 1100 (or any other mold disclosed herein) can be used in conjunction with other blow molding equipment (such as a blow molding machine, an extruder, a cutter, a pneumatic system, a heater, etc.) to make the catheter balloon. The intermediate mold portion 1120 forms an intermediate portion (such as intermediate portion 284) of a catheter balloon. The proximal mold portion 1110 and the distal mold portion 1130 can be configured to form proximal and distal end portions (such as proximal end portion 282 and distal end portion 286) of a catheter balloon, respectively. In some examples, at least one of the proximal mold portion 1110 and the distal mold portion 1130 can be separable from the intermediate mold portion 1120.

At least one of the proximal mold portion 1110, the intermediate mold portion 1120, and the distal mold portion 1130 can comprise at least one gap 1140 configured to form a radially extending protrusion (such as protrusion 292) on a portion of a catheter balloon (such as catheter balloon 218). The gap 1140 can comprise an axially elongated channel that radially extends from an inner surface of the mold 1100 towards an outer surface of the mold 1100. The gap 1140 can extend along a length of the mold 1100 in the axial direction. In some examples, the gap 1140 can extend along the entire axial length of the mold 1100. In some examples, the gap 1140 can extend along only a portion of the entire axial length of the mold 1100. In some examples, the gap 1140 can be disposed only on the distal mold portion 1130. However, in some examples, at least one gap 1140 may additionally or alternatively be formed on any of the proximal mold portion 1110 and the intermediate mold portion 1120.

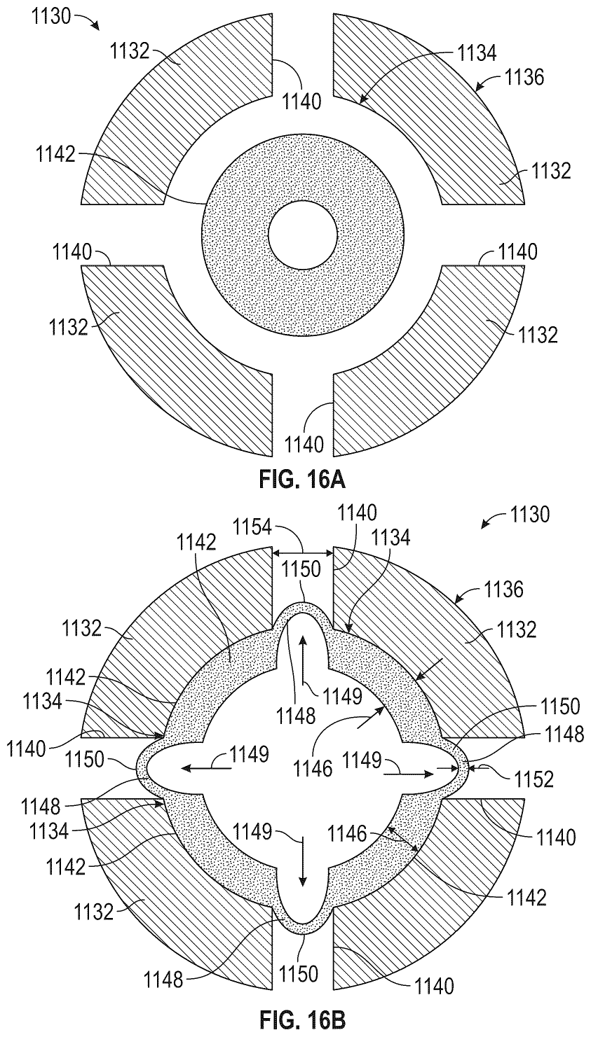

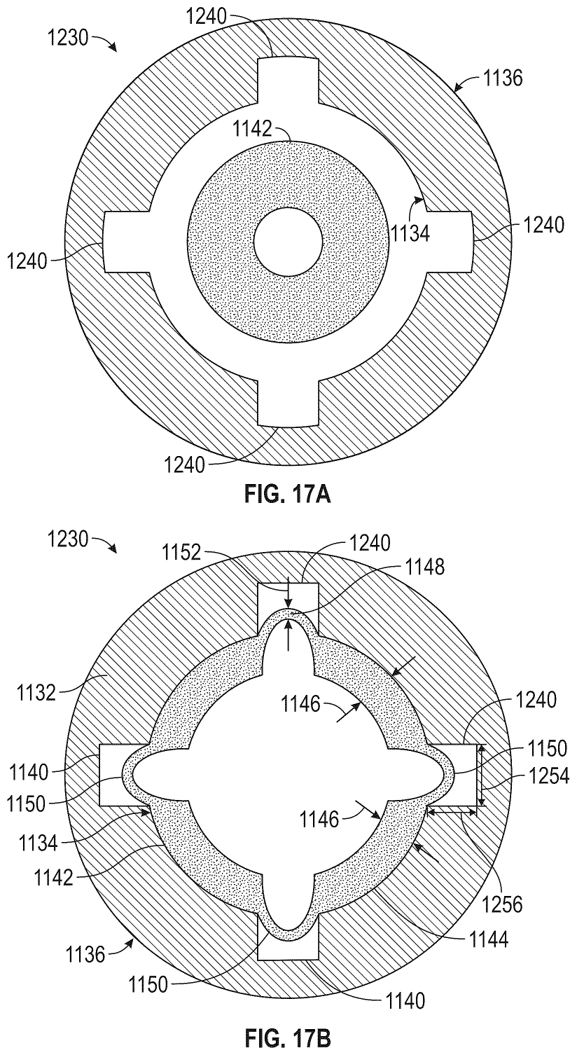

FIG. 15 shows a perspective view of the distal mold portion 1130. The distal mold portion 1130 can comprise a circumferential sidewall 1132 with an inner surface 1134 and an outer surface 1136. In some examples, the distal mold portion 1130 can comprise a hollow frustoconical portion configured to form a tapered catheter balloon end (such as distal end portion 286). In some examples, the distal mold portion 1130 can further comprise a hollow cylindrical portion 1138 extending from a distal end 1130d of the frustoconical portion that is configured to form a distal stalk (such as the distal stalk 283).

The distal mold portion 1130 can further comprise at least one gap 1140 in the sidewall 1132, wherein the gap 1140 can be configured to form a radially extending protrusion (such as protrusion 292) on a catheter balloon (such as catheter balloon 218). Although the distal mold portion 1130 illustrated in FIG. 15 comprises four gaps 1140, the distal mold portion 1130 can comprise any suitable number of gaps 1140 to form any suitable number of corresponding protrusions on a resulting catheter balloon. The gap 1140 can extend in in the radial direction from the inner surface 1134 towards the outer surface 1136. In some examples, the gap 1140 can be axially elongated to form an axially elongated protrusion (such as protrusion 292). In some examples, the gap 1140 can be configured or shaped to form a hemispheroidal or spheroidal protrusion (such as protrusion 892). In some examples, the gap 1140 can extend through the sidewall 1132 from the inner surface 1134 to the outer surface 1136. The gap 1140 can extend along the axial length of the distal mold portion 1130. In some examples, the gap 1140 can extend along the entire axial length of the distal mold portion 1130. In some examples, the gap 1140 can extend along the axial length of the distal mold portion 1130 and along an axial length of the cylindrical portion 1138.

FIGS. 16A-16B show a cross section of the distal mold portion 1130 taken along line B-B of FIG. 14 during a blow molding process to form a catheter balloon (such as catheter balloon 218). As shown in FIG. 16A, a parison 1142 of balloon material can first be inserted into the mold 1100. Then, as shown in FIG. 16B, the parison 1142 can be inflated such that a first portion 1144 of the parison 1142 contacts the inner surface 1134 of the distal mold portion 1130. The first portion 1144 (which defines a main body of the resulting catheter balloon) can have a main body thickness 1146. When the parison 1142 is inflated, a second portion 1148 of the parison 1142 can extend radially outwards (in the direction of arrows 1149) into the gap 1140 to form a protrusion 1150 having a protrusion thickness 1152. In some examples, the main body thickness 1146 can be greater than the protrusion thickness 1152. In some examples, the gap 1140 can comprise a width 1154 perpendicular to the longitudinal and radial directions. In some examples, the width 1154 of the gap 1140 can be varied to change a size, shape, or dimension (such as the protrusion thickness 1152) of the protrusion 1150.

The parison 1142 can comprise a polymer tube made of any of various suitable thermoplastics and thermoset polymers. Examples of thermoplastics include polyolefins, polyamides, such as nylon 12, nylon 11, nylon 6/12, nylon 6, and nylon 66, polyesters, polyethers, polyurethanes, polyureas, polyvinyls, polyacrylics, fluoropolymers, copolymers and block copolymers thereof, such as block copolymers of polyether and polyamide, for example, Pebax®; and mixtures thereof. Examples of thermosets include elastomers such as EPDM, epichlorohydrin, nitrile butadiene elastomers, silicones, etc. Thermosets, such as epoxies and isocyanates, can also be used. Biocompatible thermosets may also be used, and these include, for example, biodegradable polycaprolactone, poly(dimethylsiloxane) containing polyurethanes and ureas, and polysiloxanes. Any of the balloons disclosed herein can be made of one or more of any of these types of materials.

FIGS. 17A-17B illustrate a cross section of a distal mold portion 1230 during a blow molding process, according to another example. The distal mold portion 1230 can replace the mold portion 1130 of the mold 1100 to form a catheter balloon. One exemplary difference between the distal mold portion 1230 and the distal mold portion 1130 of FIGS. 16A-16B is that at least one gap 1240 extends only partially through the sidewall 1132 from the inner surface 1134 towards the outer surface 1136. The gap 1240 can have a depth 1256 extending in the radial direction from the inner surface 1134 towards the outer surface 1136 and a width 1154 perpendicular to both the longitudinal and radial directions. The depth 1256 can be less than a radial thickness of the sidewall 1132. In some examples, a size, shape, or dimension (such as the protrusion thickness 1152) of the protrusion 1150 can be changed by varying one or more dimensions of the gap 1240 (such as the width 1254 or the depth 1256).

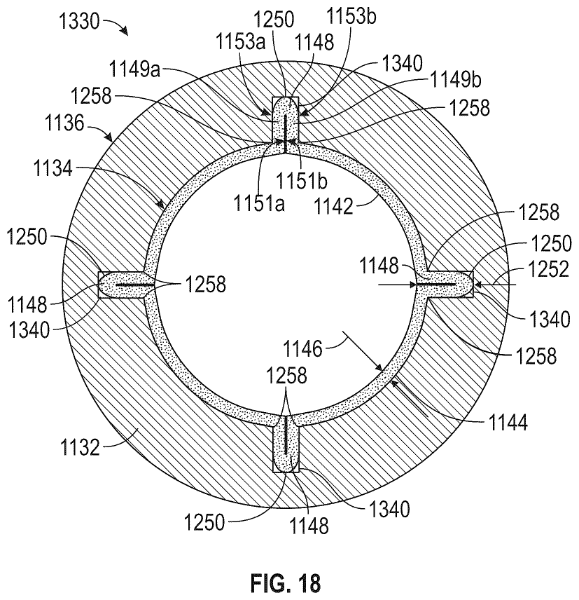

FIG. 18 illustrates a cross section of a distal mold portion 1330 during a blow molding process, according to another example. The distal mold portion 1330 can replace the mold portion 1130 of the mold 1100 to form a catheter balloon. The distal mold portion 1330 can be configured to form a distal portion of the parison 1142 into a distal end portion of a catheter balloon. One exemplary difference between the distal mold portion 1330 and the distal mold portions 1130, 1230 of FIGS. 16A-17B is that the distal mold portion 1330 can be configured to form a catheter balloon comprising at least one protrusion having a protrusion thickness greater than a thickness of a circumferentially adjacent portion (such as a main body) of the catheter balloon. Ruptures may be more likely to occur at a transition region between relatively thick portions and relatively thin portions of a catheter balloon than at a uniformly thick region of the catheter balloon. Thus, by forming a protrusion having a protrusion thickness greater than a thickness of a circumferentially adjacent portion of the catheter balloon, the catheter balloon can be configured to first rupture or tear at a transition region between the protrusion and the adjacent portion of the catheter balloon when the catheter balloon is overinflated. Transition regions on catheter balloons can be arranged to promote axial or distal tearing of the catheter balloons when the catheter balloons are overinflated, resulting in more favorable modes of rupturing that facilitate the retrieval and removal of the catheter balloons.

When the parison 1142 is inflated within the distal mold portion 1330, the first portion 1144 of the parison 1142 can contact the inner surface 1134 of the mold. The first portion 1144 can form the main body thickness 1146. The second portion 1148 of the parison 1142 can extend radially outwards into a gap 1340 to form a protrusion 1250 with a protrusion thickness 1252 oriented in the radial direction. The second portion 1148 can comprise a first half 1149a and a second half 1149b. The first half 1149a of the second portion 1148 of the parison 1142 can comprise a first inner surface 1151a and a first outer surface 1153a. The thickness of the first half 1149a (in other words, the distance from the first inner surface 1151a to the first outer surface 1153a) can be equal to the main body thickness 1146. The second half 1149b of the second portion 1148 of the parison 1142 can comprise a second inner surface 1151b and a second outer surface 1153b. The thickness of the second half 1149b (in other words, the distance from the second inner surface 1151b to the second outer surface 1153b) can be equal to the thickness of the first half 1149a and/or the main body thickness 1146. When the parison 1142 is inflated, the first inner surface 1151a and the second inner surface 1151b contact each other as the second portion 1148 enters the gap 1340 to form the protrusion 1250. In some examples, the first inner surface 1151a and the second inner surface 1151b can fuse together (becoming bonded to each other) to form the protrusion 1250 during the blow molding process.

The protrusion thickness 1252 can comprise a radial thickness of the protrusion 1250. As shown in the example illustrated in FIG. 18, the protrusion thickness 1252 can be greater than the main body thickness 1146. Similarly, the protrusion 1250 can have a thickness in the circumferential direction (perpendicular to the thickness 1252) equal to twice the thickness 1146 of the main body of the balloon. Axially extending transition regions 1258 can be formed on an outer surface of the balloon at the intersections of the first portion 1144 and the second portions 1148, wherein the transition regions 1258 may be more likely to first rupture or tear under extreme pressure from fluid introduced to inflate the resulting catheter balloon. Each transition region 1258 may extend in the axial direction along the length of the catheter balloon, such that the axial ends of the transition region 1258 are coterminous with the axial ends of the protrusion 1250. Since the transition regions 1258 may be more likely to rupture before other portions of the catheter balloon when the catheter balloon is overinflated, any ruptures or tears can propagate in the axial direction along one or more of the transition regions 1258 to result in a more preferable mode of balloon rupturing. In some examples, a transition region 1258 can have a thickness less than the thickness 1252 of the protrusion 1250 and less than the main body thickness 1146 of the parison 1142.

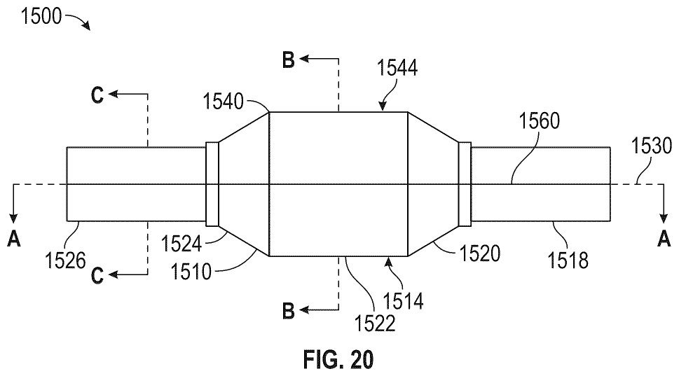

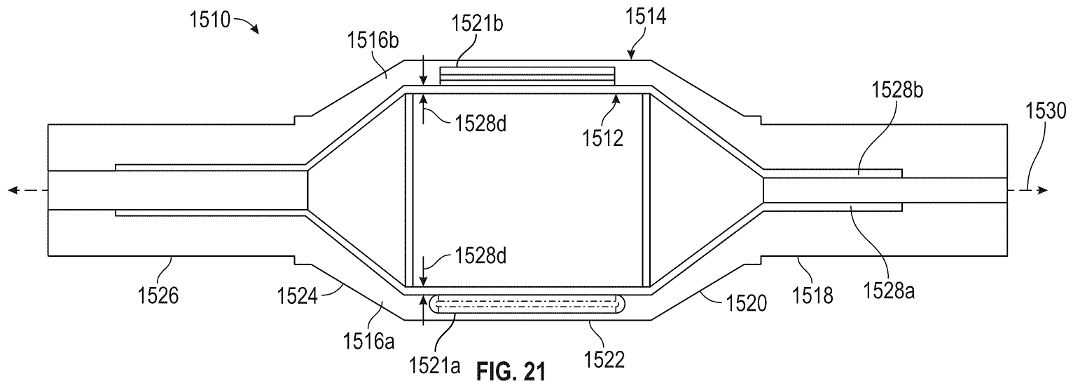

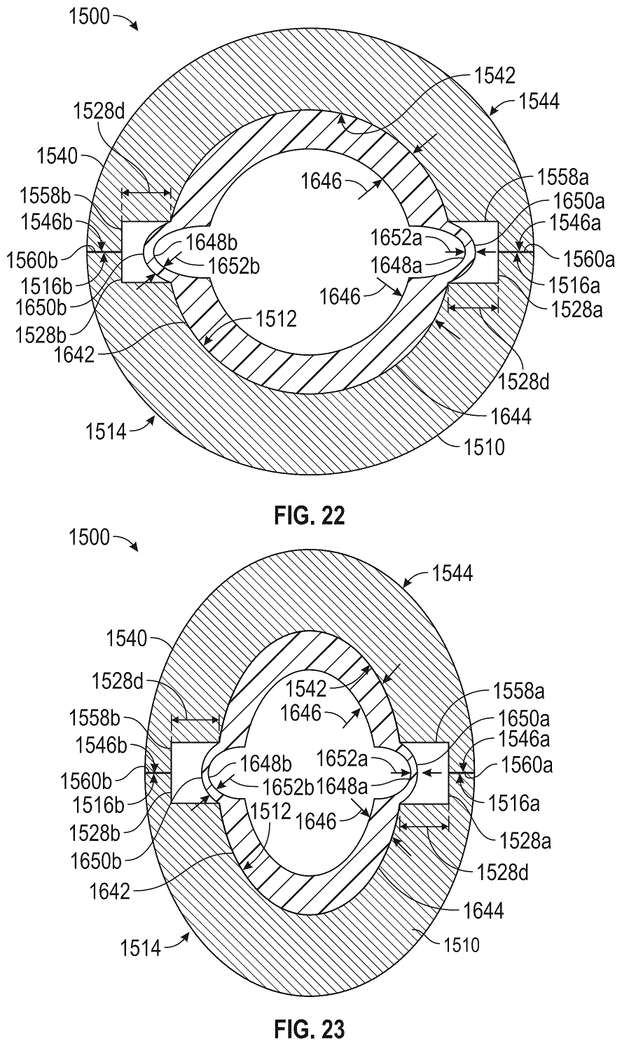

FIG. 20 shows a side view of a mold 1500 (which is also referred to herein as a “catheter balloon mold,” a “balloon mold,” and/or a “blow mold”) for forming a catheter balloon, according to one example. The mold 1500 can be configured to form any one of the catheter balloons disclosed in this application during a blow molding process. The mold 1500 can comprise a first mold portion 1510 (which is also referred to herein as a “first half”) and a second mold portion 1540 (which is also referred to herein as a “second half”). The first mold portion 1510 comprises a first inner surface 1512 (FIG. 21) and a first outer surface 1514. The second mold portion 1540 comprises a second inner surface 1542 (FIG. 22) and a second outer surface 1544 (FIG. 22). In some examples, the mold 1500 can be a clamshell mold, wherein the first mold portion 1510 and the second mold portion 1540 can be the two respective halves of the clamshell mold. In some examples, the mold 1500 can comprise additional mold portions.

When the first mold portion 1510 abuts or is coupled to the second mold portion 1540, the first mold portion 1510 and the second mold portion 1540 form a body that defines a central longitudinal axis 1530 and at least one parting line 1560 extending in an axial direction parallel to the central longitudinal axis 1530. The parting line 1560 is a plane that is formed where a first surface of the first mold portion 1510 contacts a second surface of the second mold portion 1540 or, in other words, where the first mold portion 1510 and the second mold portion 1540 meet after the mold 1500 is closed. Although only one parting line 1560 is illustrated in the present figure, some examples of the mold 1500 can comprise a plurality of parting lines.

FIG. 21 is a cross-section of the mold 1500 taken along line A-A of FIG. 20, according to one example. Since line A-A is aligned with the parting line 1560, FIG. 21 should also be understood to be a top-down view of the first mold portion 1510 and should be further understood to show the interior of the first mold portion 1510.

As shown, the first mold portion 1510 comprises a hemi-cylindrical proximal leg portion 1518, a hemi-frustoconical proximal portion 1520 distally adjacent the proximal leg portion 1518, a hemi-cylindrical intermediate portion 1522 distally adjacent the proximal portion 1520, a hemi-frustoconical distal portion 1524 distally adjacent the intermediate portion 1522, and a hemi-cylindrical distal leg portion 1526 distally adjacent the distal portion 1524. As shown, the proximal leg portion 1518, the proximal portion 1520, the intermediate portion 1522, the distal portion 1524, and the distal leg portion 1526 are formed as a unitary structure. However, any one of these features can be formed as separate components. Collectively, the aforementioned portions of the mold 1500 form a body comprising the first inner surface 1512 and the first outer surface 1514 disposed in a radially outwards-facing direction relative to the first inner surface 1512.

The first mold portion 1510 further comprises first and second parting surfaces 1516a and 1516b. When the first mold portion 1510 abuts or is coupled to the second mold portion 1540, the first and second parting surfaces 1516a and 1516b contact corresponding third and fourth parting surfaces 1546a (FIG. 22) and 1546b (FIG. 22) of the second mold portion 1540, respectively, to define first and second parting lines 1560a and 1560b (FIG. 22), respectively. In some examples, alignment features 1521a and 1521b can extend from the first and second parting surfaces 1516a and 1516b of the first mold portion 1510 and can be configured to engage corresponding alignment features 1521b and 1521a, respectively, on the second mold portion 1540. Such features can help align the first mold portion 1510 and the second mold portion 1540 when the mold portions are pressed together. For example, in the illustrated example, alignment feature 1521a comprises a slot or groove and alignment feature 1521b comprises a projection or tongue. When the first mold portion 1510 is assembled against the second mold portion 1540, the groove 1521a of the first mold portion 1510 receives the tongue 1521b of the second mold portion 1540, and the tongue 1521b of the first mold portion 1510 extends into the groove 1521a of the second mold portion 1540.

The first mold portion 1510 can further comprise one or more channels (which are also referred to herein as “gaps,” “troughs,” “slots,” “recesses,” and/or “trenches”) extending in an axial direction of the mold 1500. As shown, the first mold portion 1510 comprises first and second channels 1528a and 1528b formed on the first mold portion 1510. However, some examples of the first mold portion 1510 can comprise any number of channels (for example, one channel, three channels, four channels, etc.).

As shown, the first channel 1528a is formed circumferentially adjacent (in other words, adjacent in a circumferential direction of the mold 1500) and parallel to the first parting surface 1516a and the second channel 1528b is formed circumferentially adjacent and parallel to the second parting surface 1516b. Since parting lines are defined by their respective parting surfaces (for example, the first parting line 1560a is formed when the first parting surface 1516a is pressed against the third parting surface 1546a), the first and second channels 1528a and 1528b are also adjacent and/or parallel to the parting lines 1560a and 1560b when the first mold portion 1510 and the second mold portion 1540 are coupled together. However, in some examples, the first channel 1528a and the second channel 1528b can be formed anywhere on the first inner surface 1512 and do not need to be disposed adjacent the first and second parting surfaces 1516a and 1516b and/or the first and second parting lines 1560a and 1560b.

The first channel 1528a and the second channel 1528b can be formed substantially opposite each other on the first mold portion 1510. As shown, the first channel 1528a and the second channel 1528b are formed on substantially opposite sides of the first mold portion 1510 (for example, approximately 180 degrees apart from each other (±10 degrees) in the circumferential direction of the mold 1500). However, in some examples, the first channel 1528a and the second channel 1528b can be spaced any circumferential distance apart from each other.

As shown, the first channel 1528a and the second channel 1528b extend from the first inner surface 1512 and in a radial direction of the mold 1500 towards the first outer surface 1514. As such, each of the first and second channels 1528a and 1528b has a depth 1528d in the radial direction of the mold 1500. In some examples, at least a portion of a channel (for example, at least a portion of the first channel 1528a and/or at least a portion of the second channel 1528b, or any other channel disclosed herein) can have the depth 1528d from 0.00001″ to 0.01″, such as from 0.00005″ to 0.0065″, from 0.0001″ to 0.006″, from 0.00015″ to 0.0055″, and/or from 0.0002″ to 0.005″. As shown, the depth 1528d of the first channel 1528a and the second channel 1528b is uniform along the length of each channel, but in some examples the depth 1528d of the first channel 1528a and/or the second channel 1528b can vary along the channel's axial length. Since the radial thickness of a catheter balloon's protrusions can depend at least in part on the depth 1528d of the corresponding channels (for example, channels 1528a and 1528b), sizing the channels to have any of the aforementioned depths can beneficially result in protrusions that have a particular desired thickness.

The first channel 1528a and the second channel 1528b can extend along a partial or entire axial length of any one of the proximal leg portion 1518, the proximal portion 1520, the intermediate portion 1522, the distal portion 1524, and/or the distal leg portion 1526. For example, as shown, the first channel 1528a and the second channel 1528b extend along the entire axial length of the proximal portion 1520, the entire axial length of the intermediate portion 1522, and the entire axial length of the distal portion 1524. As further shown, the first and second channels 1528a and 1528b also extend along a partial axial length of the proximal leg portion 1518 and a partial axial length of the distal leg portion 1526. However, some examples of the first channel 1528a and/or the second channel 1528b can extend along the entire axial length of the proximal leg portion 1518 and/or the distal leg portion 1526. Furthermore, in some examples, the first channel 1528a and/or the second channel 1528b need not extend across every region or portion of the first mold portion 1510.

FIG. 22 is a cross-section of the intermediate portion 1522 of the mold 1500 taken along line B-B of FIG. 20 during a second stage of a blow molding process. At least one of the first mold portion 1510 and the second mold portion 1540 can comprise at least one channel formed thereon, wherein the channel extends in the axial direction of the mold 1500 and in the radial direction of the mold 1500 from an inner surface of the mold 1500 to an outer surface of the mold 1500. As shown, the first mold portion 1510 comprises the first channel 1528a and the second channel 1528b formed adjacent the first and second parting lines 1560a and 1560b, respectively. Each of the first channel 1528a and the second channel 1528b are shown extending the radial direction of the mold 1500 from the first inner surface 1512 towards the first outer surface 1514. The first channel 1528a and the second channel 1528b can further extend in the axial direction of the mold 1500 parallel to the first and second parting lines 1560a and 1560b, respectively. As further shown, the second mold portion 1540 comprises a third channel 1558a and a fourth channel 1558b, each of which is formed adjacent the first and second parting lines 1560a and 1560b, respectively. Each of the third channel 1558a and the fourth channel 1558b are shown extending the radial direction of the mold 1500 from the second inner surface 1542 towards the second outer surface 1544. Each of the third channel 1558a and the fourth channel 1558b can further extend in the axial direction of the mold 1500 parallel to the first and second parting lines 1560a and 1560b, respectively. When the first mold portion 1510 and the second mold portion 1540 are coupled together, the first and second parting surfaces 1516a and 1516b contact corresponding third and fourth parting surfaces 1546a and 1546b of the second mold portion 1540 to form the first and second parting lines 1560a and 1560b.

As shown, a catheter balloon can be formed by inserting a parison 1642 (which in some examples can be similar to parison 1142) into the mold 1500 and inflating the parison 1642 therein, such that the parison 1642 contacts the inner surfaces of the mold 1500. It should be understood that the act of “inflating” the parison 1642 can also involve stretching and/or heating the parison 1642 and/or the resulting catheter balloon. For example, the parison 1642 can be inflated such that a first portion 1644 of the parison 1642 contacts the first inner surface 1512 of the first mold portion 1510 and the second inner surface 1542 of the second mold portion 1540, thus forming the main body of the resulting catheter balloon. The first portion 1644 can have a first thickness 1646. When the parison 1642 is inflated, a second portion 1648a of the parison 1642 can extend in a radially outwards-facing direction into the first channel 1528a and/or the third channel 1558a to form a first protrusion 1650a having a second thickness 1652a. In some examples, the second portion 1648a of the parison 1642 can contact one or more surfaces of the first channel 1528a and/or the third channel 1558a. Similarly, a third portion 1648b of the parison 1642 can extend in the radially outward facing direction into the second channel 1528b and/or the fourth channel 1558b to form a second protrusion 1650b having a third thickness 1652b. In some examples, the third portion 1648b of the parison 1642 can contact one or more surfaces of the second channel 1528b and/or the fourth channel 1558d. As shown, the first thickness 1646 is greater than the second and third thicknesses 1652a and 1652b of each of the first and second protrusions 1650a and 1650b. As shown, the first channel 1528a, the second channel 1528b, the third channel 1558a, and the fourth channel 1558b each have the same depth 1528d. However, some examples of the channels 1528a, 1528b, 1558a, and 1558b can have different depths. For example, the first channel 1528a and the third channel 1558a can each have a first depth and the second channel 1528b and the fourth channel 1558b can each have a second depth that is different than the first depth, such that the second thickness 1652a of the second portion 1648a is different than the third thickness 1652b of the third portion 1648b.

FIG. 23 is a cross-section of the distal leg portion 1526 of the mold 1500 taken along line C-C of FIG. 20 during the second stage of the blow molding process. One exemplary difference between the cross-section of the intermediate portion 1524 shown in FIG. 22 and the presently illustrated cross-section of the distal leg portion 1526 is that the distal leg portion 1526 has an elliptical or oval cross-section. In some examples, a portion of the catheter balloon formed by the distal leg portion 1526 can be more likely to tear prior to the rest of the catheter balloon. Thus, the formation of tears in the catheter balloon can be further controlled by modifying the cross-section of a portion of the mold 1500.

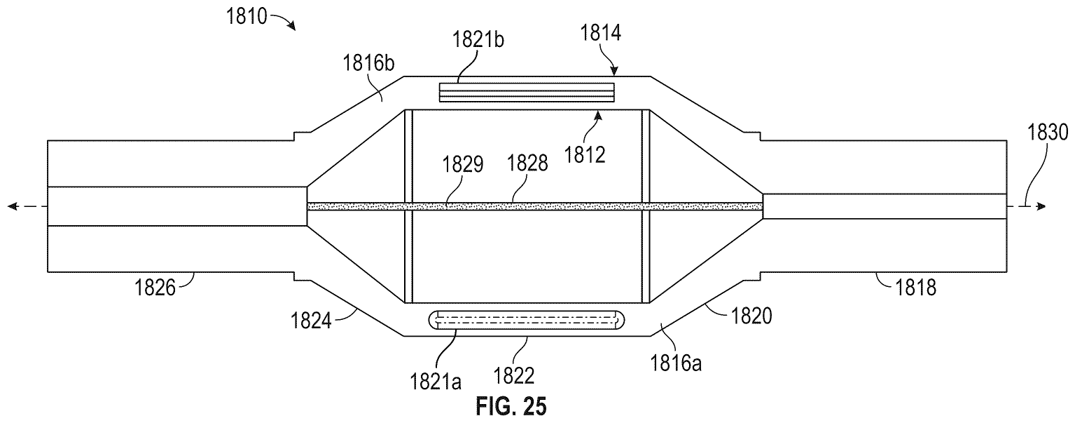

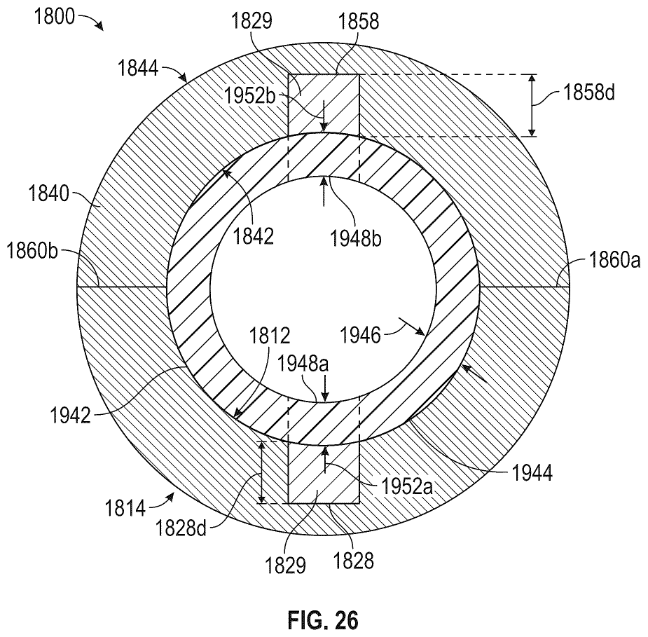

FIG. 25 is a cross-section of a mold 1800 for forming a catheter balloon. The mold 1800 can be configured to form any one of the catheter balloons disclosed in this application during a blow molding process. The mold 1800 can be similar to some examples of the mold 1500 illustrated in FIGS. 20-23, for example, in that the mold 1800 comprises a first mold portion 1810, a second mold portion 1840 (FIG. 26), and first and second parting lines 1860a and 1860b (FIG. 26) formed by the first mold portion 1810 and the second mold portion 1840, wherein the parting lines 1860a and 1860b extend in an axial direction of a central longitudinal axis 1830 of the mold 1800. The presently illustrated cross-section is taken about the parting lines 1860a and 1860b. Thus, it should be understood that FIG. 25 is also a top-down view of the first mold portion 1810.

The first mold portion 1810 can be similar to some examples of the first mold portion 1510 illustrated in FIG. 21. For example, the first mold portion 1810 can comprise a hemi-cylindrical proximal leg portion 1818, a hemi-frustoconical proximal portion 1820 distally adjacent the proximal leg portion 1818, a hemi-cylindrical intermediate portion 1822 distally adjacent the proximal portion 1820, a hemi-frustoconical distal portion 1824 distally adjacent the intermediate portion 1822, and a hemi-cylindrical distal leg portion 1826 distally adjacent the distal portion 1824. As shown, the proximal leg portion 1818, the proximal portion 1820, the intermediate portion 1822, the distal portion 1824, and the distal leg portion 1826 are formed as a unitary structure. However, any one of these features can be formed as separate components. Collectively, the aforementioned portions of the mold 1800 form a body comprising a first inner surface 1812 and the first outer surface 1814 disposed in a radially outwards-facing direction relative to the first inner surface 1812.

One exemplary difference between the first mold portion 1810 and the first mold portion 1510 illustrated in FIG. 21 is that the first mold portion 1810 is shown as comprising only one channel 1828 (which is also referred to herein as a “gap,” “trough,” “slot,” “recess,” and/or “trench”) extending in an axial direction (for example, parallel to the central longitudinal axis 1830) of the mold 1800 and from the inner surface 1812 of the first mold portion 1810 towards the outer surface 1814 of the first mold portion 1810. However, some examples of the first mold portion 1810 can comprise a plurality of channels 1828 (for example, two channels, three channels, four channels, five channels, etc.). The illustrated channel 1828 is spaced apart in a circumferential direction from a first parting surface 1816a and a second parting surface 1816b, which partially define the first and second parting lines 1860a and 1860b. Thus, the channel 1828 can also be spaced apart in a circumferential direction from the parting lines 1860a and 1860b. However, some examples of the channel 1828 can be adjacent the first parting surface 1816a, the second parting surface 1816b, and/or the first and second parting lines 1860a and 1860b.

The channel 1828 can extend along a partial or entire axial length of any one of the proximal leg portion 1818, the proximal portion 1820, the intermediate portion 1822, the distal portion 1824, and/or the distal leg portion 1826. For example, as shown, the channel 1828 extends along the entire axial length of the proximal portion 1820, the entire axial length of the intermediate portion 1822, and the entire axial length of the distal portion 1824. However, some examples of the channel 1828 can extend along at least a portion of the axial length of the proximal leg portion 1818 and/or the distal leg portion 1826. However, the channel 1828 can be disposed on any region or portion of the first mold portion 1810.

In some examples, at least a portion of the channel 1828 can extend a radial distance or depth from the inner surface 1812 of the first mold portion 1810 towards the outer surface 1814. In some examples, the depth can be from 0.00001″ to 0.01″, such as from 0.00005″ to 0.0065″, from 0.0001″ to 0.006″, from 0.00015″ to 0.0055″, and/or from 0.0002″ to 0.005″. In some examples, the depth of the channel 1828 can be uniform along its axial length, but in some examples the depth of the channel 1828 can vary along its axial length.

Another exemplary difference between the first mold portion 1810 and the first mold portion 1510 illustrated in FIG. 21 is that the first mold portion 1810 comprises a filler 1829 (represented by the stippling bounded by the horizontal lines defining the channel 1828 in FIG. 25) at least partially filling the channel 1828. In some examples, the filler 1829 can completely fill the channel 1828, such that the filler 1829 is flush with the inner surface 1812 of the first mold portion 1810. In some examples, the filler 1829 can only partially fill the channel 1828, such that a recess is formed within the channel 1828.

FIG. 26 is a cross-section of the mold 1800 of FIG. 25 during a second stage of a blow molding process, according to one example. As shown, the mold 1800 comprises the first mold portion 1810, the second mold portion 1840, and the parting lines 1860a and 1860b formed by the first mold portion 1810 and the second mold portion 1840.

As shown, the first mold portion 1810 comprises the channel 1828 (which is herein referred to as a “first channel” for clarity) spaced apart in the circumferential direction from the first and second parting lines 1860a and 1860b, wherein the first channel 1828 has a first depth 1828d, which may be equal to any depth disclosed herein (for example, depth 1528d). The first channel 1828 is shown as being completely filled with the filler 1829, such that the filler 1829 is flush with the inner surface 1812 of the first mold portion 1810. However, in some examples, the filler 1829 may only partially fill the first channel 1828, such that a recess is formed in the first channel 1828. In such examples, the filler 1829 can define a bottom surface of the first channel 1828.

As further shown, the second mold portion 1840 comprises an inner surface 1842, an outer surface 1844, and a second channel 1858 extending from the inner surface 1842 and towards the outer surface 1844. The second channel 1858 is shown as spaced apart in the circumferential direction from the first and second parting lines 1860a and 1860b, wherein the second channel 1858 has a second depth 1858d, which may be equal to any depth disclosed herein (for example, depth 1528d). However, the second channel 1858 may be adjacent the parting lines 1860a and/or 1860b in some examples. The second depth 1858d is shown as being equal to the first depth 1828d of the first channel 1828, but the first and second depths 1828d and 1858d may be different in some examples. The second channel 1858 is shown as being completely filled with the filler 1829, such that the filler 1829 is flush with the inner surface 1842 of the second mold portion 1840. However, in some examples, the filler 1829 may only partially fill the second channel 1858, such that a recess is formed in the second channel 1858. In such examples, the filler 1829 can define a bottom surface of the second channel 1858.

The first mold portion 1810 and/or the second mold portion 1840 can be formed from a material (for example, a copper alloy) having a first thermal conductivity. The filler 1829 can be formed from a material (for example, stainless steel) having a second thermal conductivity that is different than (for example, less than) the first thermal conductivity. Although the illustrated mold 1800 is shown as having the same filler 1829 in each of the first channel 1828 and the second channel 1858, some examples of the mold 1800 can comprise a first filler in the first channel 1828 and a second filler in the second channel 1858, wherein the first filler and the second filler are formed from materials having different thermal conductivities.

As shown, a parison 1942 (which in some examples can be similar to parison 1142 and or parison 1642) can be inserted into the mold 1800 and inflated such that a first portion 1944 of the parison 1942 contacts the first inner surface 1812 and the second inner surface, thus forming the main body of the resulting catheter balloon. The first portion 1944 can have a main body thickness 1946. When the parison 1942 is inflated, a second portion 1948a of the parison 1942 can extend in a radially outwards-facing direction to contact the filler 1829 disposed in the first channel 1828. Similarly, a third portion 1948b of the parison 1942 can extend in the radially outward facing direction to contact the filler 1829 disposed in the second channel 1858. As shown, the main body thickness 1946 is equal to the thickness 1952a of the second portion 1948a of the parison 1942 and is also equal to the thickness 1952b of the third portion 1948b of the parison 1942. However, any one of the thicknesses 1952a and 1952b can be less than the main body thickness 1946 in some examples.

In some examples, portions of the resulting catheter balloon corresponding to the second portion 1948a and/or the third portion 1948b of the parison 1942 can have different material properties than the portions of the resulting catheter balloon corresponding to first portion 1944 of the parison 1942. For example, the first portion 1944 of the parison 1942 can be formed into a portion of the resulting catheter balloon having a first yield strength and the second portion 1948a and/or the third portion 1948b of the parison 1942 can be formed into portions (similar to axial strips 1792) of the resulting catheter balloon which have a second, different yield strength. In some examples, the first yield strength can be greater than the second yield strength, such that the portions of the resulting catheter balloon corresponding to the second portion 1948a and the third portion 1948b of the parison 1942 can fail or tear before portions of the resulting catheter balloon corresponding to first portion 1944 of the parison 1942. In some examples, such differences in yield strengths can be caused at least in part by the differences in thermal conductivity between the first material (forming the first and second mold portions 1810 and 1840) and the second material (forming the filler 1829). For example, if the second material has a lower thermal conductivity than the first material, less heat can be transferred from the filler 1829 to the second and third portions 1948a and 1948b of the parison 1942 than from the first and second mold portions 1510 and 1540 to the first portion 1944 of the parison 1942. Such a difference in heat transfer can, for example, result in the different portions of the resulting catheter balloon having different material properties (such as yield strength, cross-linking density, etc.). In some examples, the parison 1942 in FIG. 26 can comprise a unitary body formed from one material; that is, the first, second, and third portions 1944, 1948a, and 1948b can be different sections of the same unitary body. Thus, the second and third portions 1948a and 1948b (outlined by the dashed lines) can be portions of a unitary body formed from the same material but having different material properties than the first portion 1944 of the same unitary body.

Any of the examples of balloons described above (including balloons 218, 318, 418, 518, 618, 718, 818, 918, 1018, 1418, and 1718) can be formed with respective protrusions that have a protrusion thickness that is greater than a main body thickness such that the balloon can rupture axially along a transition region. In some examples, a balloon can comprise a combination of one or more protrusions having a relatively thinner protrusion thickness relative to a main body thickness (such as shown in FIG. 4) and one or more protrusions having a relatively thicker protrusion thickness relative to a main body thickness (such as shown in FIG. 18).

### Delivery Techniques

For implanting a prosthetic valve within the native aortic valve via a transfemoral delivery approach, the prosthetic valve is mounted in a radially compressed state along the distal end portion of a delivery apparatus. The prosthetic valve and the distal end portion of the delivery apparatus are inserted into a femoral artery and are advanced into and through the descending aorta, around the aortic arch, and through the ascending aorta. The prosthetic valve is positioned within the native aortic valve and radially expanded (for example, by inflating a catheter balloon, actuating one or more actuators of the delivery apparatus, or deploying the prosthetic valve from a sheath to allow the prosthetic valve to self-expand). Alternatively, a prosthetic valve can be implanted within the native aortic valve in a transapical procedure, whereby the prosthetic valve (on the distal end portion of the delivery apparatus) is introduced into the left ventricle through a surgical opening in the chest and the apex of the heart and the prosthetic valve is positioned within the native aortic valve. Alternatively, in a transaortic procedure, a prosthetic valve (on the distal end portion of the delivery apparatus) is introduced into the aorta through a surgical incision in the ascending aorta, such as through a partial J-sternotomy or right parasternal mini-thoracotomy, and then advanced through the ascending aorta toward the native aortic valve.

For implanting a prosthetic valve within the native mitral valve via a transseptal delivery approach, the prosthetic valve is mounted in a radially compressed state along the distal end portion of a delivery apparatus. The prosthetic valve and the distal end portion of the delivery apparatus are inserted into a femoral vein and are advanced into and through the inferior vena cava, into the right atrium, across the atrial septum (through a puncture made in the atrial septum), into the left atrium, and toward the native mitral valve. Alternatively, a prosthetic valve can be implanted within the native mitral valve in a transapical procedure, whereby the prosthetic valve (on the distal end portion of the delivery apparatus) is introduced into the left ventricle through a surgical opening in the chest and the apex of the heart and the prosthetic valve is positioned within the native mitral valve.

For implanting a prosthetic valve within the native tricuspid valve, the prosthetic valve is mounted in a radially compressed state along the distal end portion of a delivery apparatus. The prosthetic valve and the distal end portion of the delivery apparatus are inserted into a femoral vein and are advanced into and through the inferior vena cava, and into the right atrium, and the prosthetic valve is positioned within the native tricuspid valve. A similar approach can be used for implanting the prosthetic valve within the native pulmonary valve or the pulmonary artery, except that the prosthetic valve is advanced through the native tricuspid valve into the right ventricle and toward the pulmonary valve/pulmonary artery.

Another delivery approach is a transatrial approach whereby a prosthetic valve (on the distal end portion of the delivery apparatus) is inserted through an incision in the chest and an incision made through an atrial wall (of the right or left atrium) for accessing any of the native heart valves. Atrial delivery can also be made intravascularly, such as from a pulmonary vein. Still another delivery approach is a transventricular approach whereby a prosthetic valve (on the distal end portion of the delivery apparatus) is inserted through an incision in the chest and an incision made through the wall of the right ventricle (typically at or near the base of the heart) for implanting the prosthetic valve within the native tricuspid valve, the native pulmonary valve, or the pulmonary artery.

In all delivery approaches, the delivery apparatus can be advanced over a guidewire previously inserted into a patient's vasculature. Moreover, the disclosed delivery approaches are not intended to be limited. Any of the prosthetic valves disclosed herein can be implanted using any of various delivery procedures and delivery devices known in the art.

### Sterilization

Any of the systems, devices, apparatuses, etc. herein can be sterilized (for example, with heat/thermal, pressure, steam, radiation, and/or chemicals, etc.) to ensure they are safe for use with patients, and any of the methods herein can include sterilization of the associated system, device, apparatus, etc. as one of the steps of the method. Examples of heat/thermal sterilization include steam sterilization and autoclaving. Examples of radiation for use in sterilization include, without limitation, gamma radiation, ultra-violet radiation, and electron beam. Examples of chemicals for use in sterilization include, without limitation, ethylene oxide, hydrogen peroxide, peracetic acid, formaldehyde, and glutaraldehyde. Sterilization with hydrogen peroxide may be accomplished using hydrogen peroxide plasma, for example.

### Simulation

The treatment techniques, methods, steps, etc. described or suggested herein or in references incorporated herein can be performed on a living animal or on a non-living simulation, such as on a cadaver, cadaver heart, anthropomorphic ghost, simulator (for example, with the body parts, tissue, etc. being simulated), etc.

### Additional Examples of the Disclosed Technology

In view of the above-described implementations of the disclosed subject matter, this application discloses the additional examples enumerated below. It should be noted that one feature of an example in isolation or more than one feature of the example taken in combination and, optionally, in combination with one or more features of one or more further examples are further examples also falling within the disclosure of this application.

Example 1. A balloon catheter comprising: a shaft having a proximal end portion and a distal end portion; and an inflatable balloon mounted on the distal end portion of the shaft, the balloon comprising: a main body having a tapered distal end portion, a tapered proximal end portion, and an intermediate portion extending therebetween; and at least one protrusion disposed only on an outer surface of the distal end portion of the main body of the balloon, the at least one protrusion extending in an axial direction of the distal end portion of the balloon and in a radially outward direction.

Example 2. The balloon catheter of any example herein, particularly example 1, wherein the main body comprises a main body sidewall with a first thickness and the protrusion comprises a protrusion sidewall with a second thickness.

Example 3. The balloon catheter of any example herein, particularly example 2, wherein the first thickness is greater than the second thickness.

Example 4. The balloon catheter of any example herein, particularly example 3, wherein the balloon is configured to first rupture at the protrusion when the balloon is overinflated.

Example 5. The balloon catheter of any example herein, particularly example 2, wherein the first thickness is less than the second thickness.

Example 6. The balloon catheter of any example herein, particularly example 5, wherein the balloon comprises a transition region disposed on the outer surface of the distal end portion and adjacent the protrusion, and wherein the balloon is configured to first rupture at the transition region when the balloon is overinflated.

Example 7. The balloon catheter of any example herein, particularly any one of examples 1-6, wherein the protrusion extends along an entire axial length of the distal end portion of the main body.

Example 8. The balloon catheter of any example herein, particularly any one of examples 1-6, wherein the protrusion extends only partially along an entire axial length of the distal end portion of the main body.

Example 9. A balloon catheter comprising: a shaft; a balloon coupled to a distal end portion of the shaft, the balloon comprising: a main body having a first thickness; and a protrusion extending radially outwards from the main body and in an axial direction along the main body, wherein the protrusion comprises a sidewall having a second thickness, wherein the first thickness is greater than the second thickness.

Example 10. The balloon catheter of any example herein, particularly example 9, wherein the balloon comprises a proximal end portion coupled to the distal end portion of the shaft, and wherein the balloon is configured such that the proximal end portion remains intact and connected to the shaft if the balloon ruptures from overinflation.

Example 11. The balloon catheter of any example herein, particularly any one of examples 9-10, wherein the balloon is configured to first rupture at the protrusion when the balloon is overinflated.

Example 12. The balloon catheter of any example herein, particularly any one of examples 9-11, wherein the balloon is inflatable between a deflated state and an inflated state.

Example 13. The balloon catheter of any example herein, particularly example 12, wherein the protrusion is configured to collapse radially inwards when the balloon is in the deflated state.

Example 14. A catheter balloon comprising: a first portion extending in a circumferential direction around the catheter balloon; and a second portion extending along a length of the first portion and in a radially outward direction, wherein the second portion is configured to rupture prior to the first portion in an axial direction under a predetermined pressure from an inflation fluid introduced into the catheter balloon.

Example 15. The catheter balloon of any example herein, particularly example 14, wherein the first portion is integrally formed with the second portion.

Example 16. The catheter balloon of any example herein, particularly any one of examples 14-15, wherein the first portion has a first radial thickness, and the second portion has a second radial thickness.

Example 17. The catheter balloon of any example herein, particularly example 16, wherein the first radial thickness is greater than the second radial thickness.

Example 18. A catheter balloon comprising: a first portion extending in a circumferential direction around the balloon and having a first wall thickness; and a second portion extending in an axial direction along a length of the first portion and in a radially outward direction, the second portion having a second wall thickness, wherein the first wall thickness is greater than the second wall thickness.

Example 19. The catheter balloon of any example herein, particularly example 18, wherein the first portion is a distal end portion of a main body of the catheter balloon.

Example 20. The catheter balloon of any example herein, particularly any one of examples 18-19, wherein the second portion extends along an entirety of the length of the first portion.

Example 21. The catheter balloon of any of claims 18-19, wherein the second portion extends only partially along an entirety of the length of the first portion.

Example 22. A catheter balloon comprising: a main body comprising a first sidewall having a first sidewall thickness; and a plurality of protrusions extending radially outwards from the main body and in an axial direction along the main body, wherein each of the plurality of protrusions comprises a second sidewall having a second sidewall thickness, wherein the first sidewall thickness is greater than the second sidewall thickness.

Example 23. The catheter balloon of any example herein, particularly example 22, wherein the plurality of protrusions are evenly spaced apart on the main body in a circumferential direction.

Example 24. The catheter balloon of any example herein, particularly example 22, wherein the plurality of protrusions are unevenly spaced apart on the main body in a circumferential direction.

Example 25. A catheter balloon comprising: a main body comprising an inner surface, an outer surface, and a first thickness therebetween; and at least one axially elongated protrusion extending radially outwards from the outer surface, wherein the axially elongated protrusion comprises a sidewall having a second thickness, wherein the first thickness is greater than the second thickness.

Example 26. The catheter balloon of any example herein, particularly example 25, wherein the axially elongated protrusion has a semicircular axial cross section.

Example 27. The catheter balloon of any example herein, particularly example 25, wherein the axially elongated protrusion has a semi-elliptical axial cross section.

Example 28. The catheter balloon of any example herein, particularly any one of examples 25-27, wherein the main body comprises a distal end portion, a tapered proximal end portion, and an intermediate portion extending therebetween.

Example 29. The catheter balloon of any example herein, particularly example 28, wherein the axially elongated protrusion extends along a length of the intermediate portion of the main body.

Example 30. The catheter balloon of any example herein, particularly example 28, wherein the axially elongated protrusion extends along a length of the distal end portion of the main body.

Example 31. The catheter balloon of any example herein, particularly example 30, wherein the axially elongated protrusion extends only along the length of the distal end portion.

Example 32. The catheter balloon of any example herein, particularly example 28, wherein the main body further comprises a distal stalk extending in a distal direction from the distal end portion, and wherein the axially elongated protrusion extends along a length of the distal stalk.

Example 33. A catheter balloon comprising: a main body comprising an inner surface, an outer surface, and a first sidewall thickness therebetween; and a plurality of weak spots extending radially outwards from the outer surface and disposed along a length of the main body, wherein the each of the plurality of weak spots comprises a sidewall having a second sidewall thickness, wherein the first sidewall thickness is greater than the second sidewall thickness.

Example 34. The catheter balloon of any example herein, particularly example 33, wherein the catheter balloon is configured to first rupture at one of the plurality of weak spots when the balloon is overinflated.

Example 35. The catheter balloon of any example herein, particularly any one of examples 33-34, wherein the plurality of weak spots are axially aligned with each other.

Example 36. The catheter balloon of any example herein, particularly any one of examples 33-35, wherein at least one of the plurality of weak spots has a hemispheroidal shape.

Example 37. The catheter balloon of any example herein, particularly any one of examples 33-36, wherein at least one of the plurality of weak spots has a hemispherical shape.

Example 38. The catheter balloon of any example herein, particularly any one of examples 33-37, wherein the plurality of weak spots are disposed at a distal end portion of the main body.

Example 39. The catheter balloon of any example herein, particularly example 38, wherein the plurality of weak spots are disposed only along the distal end portion of the main body.

Example 40. A method of forming a catheter balloon comprising: inserting a parison into a mold comprising: an inner surface and an outer surface; and at least one gap extending in an axial direction of the mold and in a radial direction from the inner surface towards the outer surface; and blow molding the parison to form the catheter balloon, wherein blow molding the parison inflates the parison within the mold and causes a first portion of the parison to contact the inner surface of the mold and a second portion of the parison to extend into the gap to form a protrusion having a wall thickness that is less than a wall thickness of the first portion.

Example 41. The method of any example herein, particularly example 40, wherein the gap extends from the inner surface to the outer surface.

Example 42. The method of any example herein, particularly example 40, wherein the gap extends only partially from the inner surface to the outer surface.

Example 43. The method of any example herein, particularly example 42, wherein the gap comprises a depth extending in the radial direction and a width extending in a direction perpendicular to both the axial direction and the radial direction.

Example 44. The method of any example herein, particularly example 43, wherein the wall thickness of the protrusion can be changed by varying at least one of the depth and the width of the gap.

Example 45. A mold for a catheter balloon comprising: a first mold portion comprising a first inner surface and a first outer surface, a second mold portion comprising a second inner surface and a second outer surface, wherein the first mold portion and the second mold portion form a parting line extending in an axial direction of the mold when the first mold portion and the second mold portion are coupled together; and a channel formed on the first mold portion parallel to and adjacent the parting line, wherein the channel extends in a radial direction from the first inner surface towards the first outer surface.

Example 46. The mold of any example herein, particularly example 45, wherein the channel can have a depth in the radial direction from 0.0002″ to 0.005″.

Example 47. The mold of any example herein, particularly any one of examples 45-46, wherein the first mold portion can comprise a hemi-cylindrical intermediate portion, wherein the channel can extend at least partially along an axial length of the intermediate portion adjacent the parting line.

Example 48. The mold of any example herein, particularly example 47, wherein the channel can extend along the entire axial length of the intermediate portion.

Example 49. The mold of any example herein, particularly any one of examples 45-48, wherein the first mold portion can comprise a hemi-frustoconical distal portion distally adjacent the intermediate portion, wherein the channel can extend along an axial length of the distal portion adjacent the parting line.

Example 50. The mold of any example herein, particularly example 49, wherein the channel can extend along the entire axial length of the distal portion.

Example 51. The mold of any example herein, particularly any one of examples 45-50, wherein the first mold portion can comprise a hemi-cylindrical distal leg portion extending from the distal portion, wherein the channel can extend along an axial length of the distal leg portion adjacent the parting line.

Example 52. The mold of any example herein, particularly any one of examples 45-51, wherein the channel can be a first channel.

Example 53. The mold of any example herein, particularly example 52, wherein the first channel can be disposed at a first circumferential location on the first mold portion, and wherein the mold further can comprise a second channel formed at a second circumferential location on the first mold portion, the second channel extending parallel to the parting line and in the radial direction from the first inner surface towards the first outer surface.

Example 54. The mold of any example herein, particularly example 53, wherein the first circumferential location can be 180 degrees apart from the second circumferential location.

Example 55. The mold of any example herein, particularly example 52, can further comprise a second channel formed on the second mold portion adjacent both the parting line and the first channel, wherein the second channel can extend in the radial direction from the second inner surface towards the second outer surface.

Example 56. A mold comprising: a body comprising an inner surface and an outer surface, the body being formed from a first material having a first thermal conductivity; a trough formed on the inner surface of the body, the trough extending from the inner surface towards the outer surface and in an axial direction of the mold; and a filler at least partially filling the trough, wherein the filler is formed of a second material having a second thermal conductivity that is different than the first thermal conductivity.

Example 57. The mold of any example herein, particularly example 56, wherein the first thermal conductivity can be greater than the second thermal conductivity.

Example 58. The mold of any example herein, particularly any one of examples 56-57, wherein the first material can comprise a copper alloy.

Example 59. The mold of any example herein, particularly any one of examples 56-58, wherein the second material can comprise stainless steel.

Example 60. The mold of any example herein, particularly any one of examples 56-59, wherein the trough can extend 0.0002″ to 0.005″ from the inner surface towards the outer surface.

Example 61. The mold of any example herein, particularly any one of examples 56-60, wherein the filler can completely fill the trough.

Example 62. The mold of any example herein, particularly example 61, wherein the filler can be flush with the inner surface of the body.

Example 63. The mold of any example herein, particularly any one of examples 56-60, wherein the can filler only partially fill the trough.

Example 64. The mold of any example herein, particularly any one of examples 56-63, wherein the body can comprise a first half and a second half, and wherein the first half and the second half of the body can form a parting line extending in an axial direction of the mold when the first half and the second half are coupled together.

Example 65. The mold of any example herein, particularly example 64, wherein the trough can be disposed directly adjacent the parting line.

Example 66. The mold of any example herein, particularly example 64, wherein the trough and the parting line can be spaced apart in a circumferential direction.

Example 67. A method of forming a catheter balloon comprising: inserting a parison into a mold and comprising: a parting line extending in an axial direction of the mold, an inner surface and an outer surface, and at least one channel extending parallel to and adjacent the parting line and in a radial direction from the inner surface towards the outer surface; and blow molding the parison inflates the parison within the mold and causes a first portion of the parison to contact the inner surface of the mold and a second portion of the parison to extend into the channel to form a protrusion having a wall thickness that is less than a wall thickness of the first portion.

Example 68. The method of any example herein, particularly example 67, wherein the channel can comprise a bottom surface, and wherein the second portion of the parison can contact the bottom surface of the channel.

Example 69. The method of any example herein, particularly example 68, wherein a radial distance between the inner surface of the mold and the bottom surface of the channel can be from 0.0002″ to 0.005″.

Example 70. The method of any example herein, particularly any one of examples 68-69, wherein the mold can be formed from a first material, the bottom surface of the channel can be formed from a second material, and the first material can have a higher thermal conductivity than the second material.

Example 71. The method of any example herein, particularly any one of examples 67-70, wherein the mold can comprise a cylindrical intermediate portion, and wherein the channel can extend along an axial length of the intermediate portion.

Example 72. The method of any example herein, particularly any one of examples 67-71, wherein the mold can comprise a tapered distal portion, and wherein the channel can extend along an axial length of the tapered distal portion.

Example 73. The method of any example herein, particularly any one of examples 67-72, wherein the mold can comprise a distal leg portion, and wherein the channel can extend along an axial length of the distal leg portion.

Example 74. The method of any example herein, particularly example 73, wherein the distal leg portion can have an oval cross-section.

Example 75. A catheter balloon of any example herein, particularly any one of examples 1-44, wherein the catheter balloon can be sterilized.

The features described herein with regard to any example can be combined with other features described in any one or more of the other examples, unless otherwise stated. For example, any one or more of the features of one catheter balloon can be combined with any one or more features of another catheter balloon. As another example, any one or more features of one balloon catheter can be combined with any one or more features of another balloon catheter. As a third example, any one or more features of one catheter balloon mold can be combined with any one or more features of another catheter balloon mold.

In view of the many possible ways in which the principles of the disclosure may be applied, it should be recognized that the illustrated configurations depict examples of the disclosed technology and should not be taken as limiting the scope of the disclosure nor the claims. Rather, the scope of the claimed subject matter is defined by the following claims and their equivalents.

We claim:

1. A balloon catheter comprising:
a shaft having a proximal end portion and a distal end portion; and
an inflatable balloon mounted on the distal end portion of the shaft, the balloon comprising:
a main body having a tapered distal end portion, a tapered proximal end portion, and an intermediate portion extending therebetween; and
at least one protrusion disposed only on an outer surface of the distal end portion of the main body of the balloon, the at least one protrusion extending in an axial direction of the distal end portion of the balloon and in a radially outward direction;
wherein the balloon is configured to rupture at the protrusion when the balloon is overinflated.

2. The balloon catheter of claim 1, wherein the main body comprises a main body sidewall with a first thickness and the protrusion comprises a protrusion sidewall with a second thickness.

3. The balloon catheter of claim 1, wherein the main body comprises a main body sidewall with a first thickness and the protrusion comprises a protrusion sidewall with a second thickness, and the first thickness is greater than the second thickness.

4. The balloon catheter of claim 1 wherein the balloon is configured to first rupture at the protrusion when the balloon is overinflated.

5. The balloon catheter of claim 1, wherein the balloon comprises a transition region disposed on the outer surface of the tapered distal end portion and adjacent the protrusion, and wherein the balloon is configured to first rupture at the transition region when the balloon is overinflated.

6. The balloon catheter of claim 1, wherein the protrusion extends along an entire axial length of the distal end portion of the main body.

7. The balloon catheter of claim 1, wherein the protrusion extends only partially along an entire axial length of the distal end portion of the main body.

8. The balloon catheter of claim 1, wherein the at least one protrusion comprises a plurality of protrusions.

9. The balloon catheter of claim 8 wherein the plurality of protrusions are evenly spaced apart on the outer surface in a circumferential direction.

10. A mold for a catheter balloon comprising:
a first mold portion comprising a first inner surface and a first outer surface;
a second mold portion comprising a second inner surface and a second outer surface, wherein the first mold portion and the second mold portion form a parting line extending in an axial direction of the mold when the first mold portion and the second mold portion are coupled together; and
a channel formed on the first mold portion parallel to and adjacent the parting line, wherein the channel extends in a radial direction from the first inner surface towards the first outer surface.

11. The mold of claim 10, wherein the first mold portion comprises a hemi-cylindrical intermediate portion, wherein the channel extends at least partially along an axial length of the intermediate portion adjacent the parting line.

12. The mold of claim 11, wherein the channel extends along the entire axial length of the intermediate portion.

13. The mold of claim 10, wherein the first mold portion comprises a hemi-frustoconical distal portion distally adjacent the intermediate portion, wherein the channel extends along an axial length of the distal portion adjacent the parting line.

14. The mold of claim 13, wherein the channel extends along the entire axial length of the distal portion.

15. The mold of claim 10, wherein:
the first mold portion is formed from a first material having a first thermal conductivity,
the mold further comprises a filler at least partially filling the channel, and
the filler is formed of a second material having a second thermal conductivity that is different than the first thermal conductivity.

16. The mold of claim 15, wherein the first thermal conductivity is greater than the second thermal conductivity.

17. The mold of claim 10, wherein the channel is disposed directly adjacent the parting line.

18. A method of forming a catheter balloon comprising:
inserting a parison into a mold comprising:
a parting line extending in an axial direction of the mold;
an inner surface and an outer surface; and
at least one channel extending in a radial direction from the inner surface towards the outer surface; and
blow molding the parison to form the catheter balloon, wherein blow molding the parison inflates the parison within the mold and causes a first portion of the parison to contact the inner surface of the mold and a second portion of the parison to extend into the channel to form a protrusion having a wall thickness that is less than a wall thickness of the first portion.

19. The method of claim 18, wherein the channel comprises a bottom surface, and wherein the second portion of the parison contacts the bottom surface of the channel.

20. The method of claim 19, wherein a body of the mold is formed from a first material, the bottom surface of the channel is formed from a second material, and the first material has a higher thermal conductivity than the second material.
